SEQUENCE LISTING (4 sequences)

SEQ ID NO: 1 - protein, 311 aa, Pseudomonas stutzeri
MNKNKTLLALCLGSAMALAGQAHAATGSGYTATKYPIVLAHGMLGFDSLLGIDYWYGIPSALRRDGAQVYVTEVSQLNTSELRGEELLAQVEEIVAISGKPKVNLVGHSHGGPTVRYVAGVRPDLIASVTSVGAPHKGSDVADLIRKIPEGSSGEAIIAGLVNAMGSFINFVSGSSSTAPQDSLGSLESLNSEGAARFNAKFPQGIPTTACGEGAYRVNGVRYYSWSGTSPLTNPLDISDAMMGAGALAFSGPNDGLVGRCSSHLGMVIRDNYRMNHLDEVNQFMGLTSLFETDPVSVYRQHANRLKNAGL

SEQ ID NO: 2 - protein, 364 aa, Burkholderia cepacia
MARTMRSRVVAGAVACAMSIAPFAGTTAVMTLATTHAAMAATAPAAGYAATRYPIILVHGLSGTDKYAGVLEYWYGIQEDLQQNGATVYVANLSGFQSDDGPNGRGEQLLAYVKTVLAATGATKVNLVGHSQGGLSSRYVAAVAPDLVASVTTIGTPHRGSEFADFVQDVLAYDPTGLSSSVIAAFVNVFGILTSSSHNTNQDALAALQTLTTARAATYNQNYPSAGLGAPGSCQTGAPTETVGGNTHLLYSWAGTAIQPTLSVFGVTGATDTSTLPLVDPANVLDLSTLALFGTGTVMINRGSGQNDGLVSKCSALYGKVLSTSYKWNHLDEINQLLGVRGAYAEDPVAVIRTHANRLKLAGV

SEQ ID NO: 3 - protein, 364 aa, Pseudomonas sp. KWI-56
MARTMRSRVVAGAVACAMSIAPFAGTTAVMTLATTHAAMAATAPADGYAATRYPIILVHGLSGTDKYAGVVEYWYGIQEDLQQNGATVYVANLSGFQSDDGANGRGEQLLAYVKTVLAATGATKVNLVGHSQGGLTSRYVAAVAPDLVASVTTIGTPHRGSEFADFVQNVLAYDPTGLSSSVIAAFVNVFGILTSSSHNTNQDALAALQTLTTARAATYNQNYPSAGLGAPGSCQTGAPTETVGGNTHLLYSWAGTAIQPTLSVFGITGATDTSTVPLVDLANVLDPSTLALFGTGTVMINRGSGQNDGLVSKCSALYGKVLSTSYKWNHLDEINQLLGVRGAYAEDPVAVIRTHANRLKLAGV

SEQ ID NO: 4 - protein, 347 aa, Burkholderia sp. TJI49
MSVAPFAGTAALTMAATTRAALAATAPADAYAATRYPIILVHGLTGTDKYAGVLDYWYGIREDLQQHGATVYVANLSGFQSDDGPNGRGEQLLAYVKTVLAATGAAKVNLIGHSQGGLTSRYVAAVAPDLVASVTTIGTPHRGSEFADFVQGVLATDPTGLSSSVIAAFVNVFGILTSSTHNTNQDALAALQTLTTARAAAYNQNYPSAGLGAPGSCQTGAPTETVGGNTHLLYSWAGTAIQPTVSAFGVTGAADTSTIPLVDPANALDLSTLALLGTGTVMINRGSGQNDGLVSKCSALYGKVLSTSYKWNHLDEINQLLGVRGAYAEDPLAVIRTHANRLKLAGV

(19) United States
(12) Patent Application Publication
(10) Pub. No.: US 2023/0220427 A1
(43) Pub. Date: Jul. 13, 2023

(54) PROCESSES RELATED TO FORMATION OF ARYLCYCLOPROPYL CARBOXYLIC ACIDS

(71) Applicant: CORTEVA AGRISCIENCE LLC, INDIANAPOLIS, IN (US)

(72) Inventors: PABLO JOSE CABRERA VENTURA, ZIONSVILLE, IN (US); NAKYEN CHOY, CARMEL, IN (US); STEFFEN N. GOOD, INDIANAPOLIS, IN (US); FANGZHENG LI, CARMEL, IN (US); ELIZABETH O. MCCUSKER, CARMEL, IN (US); PATRICK JAMES NELSON, INDIANAPOLIS, IN (US); JEFFREY NISSEN, INDIANAPOLIS, IN (US); MATTHEW ARNEL ROBINSON, INDIANAPOLIS, IN (US); TAY ROSENTHAL, INDIANAPOLIS, IN (US)

(73) Assignee: CORTEVA AGRISCIENCE LLC, INDIANAPOLIS, IN (US)

(21) Appl. No.: 17/996,640

(22) PCT Filed: Apr. 21, 2021
(86) PCT No.: PCT/US2021/028280

§ 371 Date: Oct. 20, 2022

Related U.S. Application Data
(60) Provisional application No. 63/014,758, filed on Apr. 24, 2020.

(51) Int. Cl. C12P 7/40; C07C 61/08; C07C 51/305; C12N 9/18

(52) U.S. Cl. CPC C12P 7/40; C07C 61/08; C07C 51/305; C12N 9/18; A01N 53/00

(57) ABSTRACT

This disclosure relates to processes to form arylcyclopropyl carboxylic acids useful in forming molecules having pesticidal utility against pests in Phyla Arthropoda, Mollusca, and Nematoda.

### CROSS REFERENCE TO RELATED APPLICATIONS

This application claims the benefit of U.S. Provisional Patent Application Ser. No. 63/014,758 filed Apr. 24, 2020, which is expressly incorporated by reference herein.

### FIELD OF THIS DISCLOSURE

This disclosure relates to processes to form arylcyclopropyl carboxylic acids useful in forming molecules having pesticidal utility against pests in Phyla Arthropoda, Mollusca, and Nematoda.

### BACKGROUND OF THIS DISCLOSURE

Formation of arylcyclopropyl carboxylic acids has been disclosed in applications WO/2016/168056; WO/2016/168058; WO/2016/168059; WO/2018/071320; and WO/2018/071327.

### REFERENCE TO SEQUENCE LISTING SUBMITTED ELECTRONICALLY

The official copy of the sequence listing is submitted electronically via EFS-Web as an ASCII formatted sequence listing with a file named 81306_ST25.txt created on Apr. 19, 2021 and having a size of 12 kilobytes and is filed concurrently with the specification. The sequence listing comprised in this ASCII formatted document is part of the specification and is herein incorporated by reference in its entirety.

### DEFINITIONS USED IN THIS DISCLOSURE

The examples given in these definitions are not exhaustive and must not be construed as limiting this disclosure. It is understood that a substituent should comply with chemical bonding rules and steric compatibility constraints in relation to the particular molecule to which it is attached. These definitions are only to be used for the purposes of this disclosure.

The term “alkoxy” means an alkyl further consisting of a carbon-oxygen single bond, for example, methoxy, ethoxy, propoxy, isopropoxy, butoxy, isobutoxy, and tert-butoxy.

The term “alkyl” means an acyclic, saturated, branched, or unbranched, substituent consisting of carbon and hydrogen, for example, methyl, ethyl, propyl, isopropyl, n-butyl, sec-butyl, isobutyl, and tert-butyl.

The term “aryl” means a cyclic, aromatic substituent consisting of hydrogen and carbon, for example, phenyl, naphthyl, and biphenyl.

The term “halo” means fluoro, chloro, bromo, and iodo.

The term “haloalkoxy” means an alkoxy further consisting of, from one to the maximum possible number of identical or different, halos, for example, fluoromethoxy, trifluoromethoxy, 2,2-difluoropropoxy, chloromethoxy, trichloromethoxy, 1,1,2,2-tetrafluoroethoxy, and pentafluoroethoxy.

The term “haloalkyl” means an alkyl further consisting of, from one to the maximum possible number of, identical or different, halos, for example, fluoromethyl, trifluoromethyl, 2,2-difluoropropyl, chloromethyl, trichloromethyl, and 1,1,2,2-tetrafluoroethyl.

The term “hydroxyalkyl” means an alkyl containing one or more hydroxy groups, for example, hydroxymethyl, hydroxyethyl, hydroxyisobutyl, 1,3-dihydroxybutyl, and 1,3,5-trihydroxyhexyl.

### DETAILED DESCRIPTION OF THIS DISCLOSURE

The following are processes related to the formation of arylcyclopropyl carboxylic acids.

In embodiment one of Scheme One

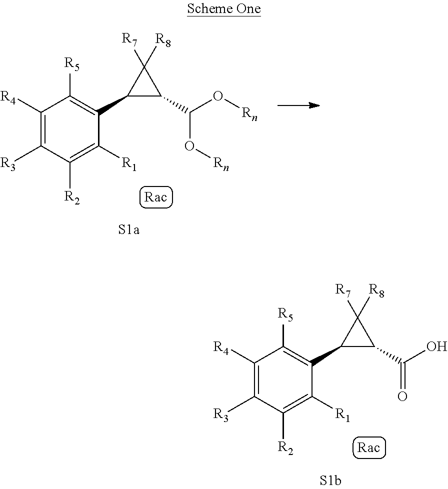

(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H;

(b) R₇ and R₈ are each independently F, Cl, Br, or I; and

(c) (1) each Rₙ is independently a (C₁-C₆)alkyl, or
(2) both Rₙ form a (C₂-C₆)alkyl link between the two oxygen atoms.

In embodiment two of Scheme One R₂ and R₄ are CF₃; R₁, R₃, and R₅, are H; R₇ and R₈ are Cl; and each Rₙ is C₂H₅. This molecule (“S1a-1”) is represented as follows.

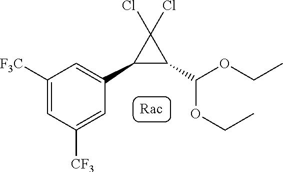 [SMILES: CCOC(OCC)[C@@H]1[C@@H](c2cc(C(F)(F)F)cc(C(F)(F)F)c2)C1(Cl)Cl]

### trans-rac-1-(2,2-dichloro-3-(diethoxymethyl)cyclopropyl)-3,5-bis(trifluoromethyl)benzene

The reaction in Scheme One is conducted in the presence of an oxidizing agent that oxidizes S1a to S1b. In other words, functionally the oxidizing agent oxidizes —C(ORₙ)₂ or —C(—O((C₂-C₆)alkyl)O—) to —C(═O)OH. Examples of oxidizing agents are oxygen (O₂), sodium hypochlorite (NaOCl), ozone (O₃), hydrogen peroxide (H₂O₂), organic peracids (—OOH), and other inorganic oxidants, such as, potassium peroxymonosulfate, potassium persulfate, potassium hydrogen peroxymonosulfate sulfate (a triple salt with the formula 2KHSO₅·KHSO₄·K₂SO₄ [CAS 70693-62-8] available from E.I. du Pont de Nemours and Company or its affiliates as OXONE®, a registered trademark of E.I. du Pont de Nemours and Company or its affiliates). In general, about 0.1 moles to about 3 moles of oxidizing agent per mole of S1a, more preferably, about 0.5 moles to about 1.5 moles of oxidizing agent per mole of S1a may be used. In general, when using hydrogen peroxide (H₂O₂) solutions a concentration of about 1% w/w to about 70% w/w may be used, however, currently, about 20% w/w to about 50% w/w is preferred. Mixtures of oxidizing agents may also be used.

The reaction in Scheme One is conducted in the presence of a polar solvent. Examples of polar solvents are polar aprotic solvents and polar protic solvents. Examples of polar aprotic solvents are ethyl acetate, tetrahydrofuran (“THF”), dichloromethane, acetone, acetonitrile (“ACN”), dimethylformamide (“DMF”), and dimethyl sulfoxide (“DMSO”). Examples of polar protic solvents are acetic acid (“AcOH”), n-butanol (“n-BuOH”), isopropanol (“i-PrOH”), n-propanol (“n-PrOH”), ethanol (“EtOH”), methanol (“MeOH”), formic acid (“HCOOH”), tert-butyl alcohol (“t-BuOH”), and water (“H₂O”). Optionally, mixtures of such polar solvents may be used, and examples are indicated below in Table S1-MR.

TABLE S1-MR

| | Examples of |
|---|---|
| Mixtures | Molar Ratios |
| H₂O:AcOH; H₂O:HCOOH; H₂O:ACN; H₂O:DMF; | 1:10; 1:5; 1:3; |
| H₂O:MeOH; H₂O:EtOH; H₂O:t-BuOH; n-BuOH:AcOH; | 1:1; 10:1; 5:1; |
| i-PrOH:AcOH; n-PrOH:AcOH; EtOH:AcOH; | or 3:1 |
| MeOH:AcOH; t-BuOH:AcOH; n-BuOH:HCOOH; | |
| i-PrOH:HCOOH; n-PrOH:HCOOH; EtOH:HCOOH; | |
| MeOH:HCOOH; t-BuOH:HCOOH; n-BuOH:ACN; | |
| i-PrOH:ACN; n-PrOH:ACN; EtOH:ACN; MeOH:ACN; | |
| t-BuOH:ACN; ACN:AcOH | |

The reaction in Scheme One may be conducted at ambient temperatures and pressures. However, higher and lower temperatures and pressures may be used. Currently, temperatures from about 0° C. to about 80° C. may be used, preferably temperatures from about 20° C. to about 60° C. may be used. Currently, pressures from about 10 kilopascal (kPa) to about 1000 kPa may be used, preferably pressures from about 50 kPa to about 150 kPa may be used.

Optionally, an acid catalyst and water may be used to promote the conversion of an acetal to an aldehyde which can then undergo oxidation to an acid. Suitable examples of acid catalysts are organic acids (acetic acid, trifluoroacetic acid, formic acid, methanesulfonic acid, p-toluenesulfonic acid, citric acid), inorganic acids such as hydrogen chloride or hydrochloric acid (HCl), silico-aluminates (zeolites, alumina, silico-alumino-phosphate), sulfated zirconia (sulfated zirconium(IV) oxide), and many transition metal oxides (titanium, zirconium, and niobium). Preferably, polystyrene based ion exchange resins with strongly acidic sulfonic groups are used, an example of which is Amberlyst® 15 (CAS Number 39389-20-3). (Amberlyst is a registered trademark of The Dow Chemical Company or an affiliated company of Dow.) Even more preferred, sulfuric acid (H₂SO₄) is used. In general, the molar ratio of acid to oxidant can range from 1:4 to 1:400, more preferably from 1:20 to 1:200. Mixture of acids can be used.

In embodiment one of Scheme Two

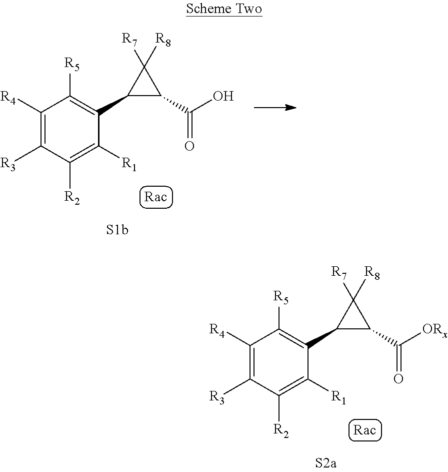

(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H;

(b) R₇ and R₈ are each independently F, Cl, Br, or I; and

(c) Rₓ is (C₁-C₆)alkyl or (C₁-C₆)hydroxyalkyl.

In embodiment two of Scheme Two R₂ and R₄ are CF₃; R₁, R₃, and R₅ are H; R₇ and R₈ are Cl; and Rₓ is CH₃. This molecule (“S2a-1”) is represented as follows.

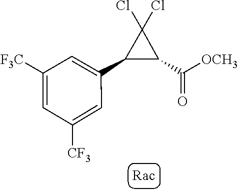 [SMILES: COC(=O)[C@@H]1[C@@H](c2cc(C(F)(F)F)cc(C(F)(F)F)c2)C1(Cl)Cl]

### trans-rac-methyl 3-(3,5-bis(trifluoromethyl)phenyl)-2,2-dichlorocyclopropane-1-carboxylate

In embodiment three of Scheme Two R₂ and R₄ are CF₃; R₁, R₃, and R₅ are H; R₇ and R₈ are Cl; and Rₓ is CH₂CH₃. This molecule (“S2a-6”) is represented as follows.

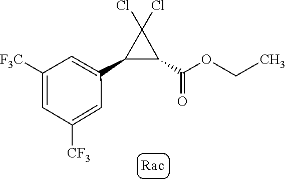 [SMILES: CCOC(=O)[C@@H]1[C@@H](c2cc(C(F)(F)F)cc(C(F)(F)F)c2)C1(Cl)Cl]

### trans-rac-ethyl 3-(3,5-bis(trifluoromethyl)phenyl)-2,2-dichlorocyclopropane-1-carboxylate

The reaction in Scheme Two is conducted in the presence of an acid that in the presence of an alcohol promotes the esterification of S1b to afford S2a. Examples of acids are sulfuric acid (H₂SO₄), p-toluenesulfonic acid monohydrate, methanesulfonic acid, scandium(III) triflate, and other acidic resins, such as Amberlyst® 15, Nafion™ (perfluorinated resin (CAS Number: 31175-20-9); Nafion™ is a trademark of The Chemours Company FC, LLC), as well as mixtures thereof. In general, about 0.001 moles to about 5 moles of acid per mole of S1b, more preferably, about 0.05 moles to about 0.5 moles of acid per mole of S1b may be used.

The reaction in Scheme Two is conducted in the presence of a (C₁-C₆)alcohol. Examples of (C₁-C₆)alcohols are n-butanol, isopropanol, n-propanol, ethanol, methanol, ethylene glycol, tert-butyl alcohol, and mixtures thereof. Optionally, mixtures of such alcohols may be used in a large variety of molar ratios, and in the presence of a solvent such as, toluene, carbon tetrachloride, benzene, diethyl ether, hexane, heptane, and dichloromethane. For example, for two component mixtures, ratios of 1:1, 1:5; 1:10, 1:50, and 1:100 may be used.

The reaction in Scheme Two may be conducted at ambient temperatures and pressures. However, higher or lower temperatures and pressures may be used. Currently, temperatures from about 0° C. to about 100° C. may be used; preferably temperatures from about 50° C. to about 70° C. may be used. Currently, pressures from about 10 kPa to about 1000 kPa may be used; preferably pressures from about 50 kPa to about 150 kPa may be used.

Any organic or inorganic drying agent may be used to remove water generated by the reaction. About 0.1 to about 5 moles of drying agent may be used per mole of S1b; preferably, about 0.2 to about 2 moles of drying agent may be used per mole of S1b. It is preferred to use an orthoester as a drying agent, especially those of formula [R_{y1}C(OR_{y2})₃] where R_{y1} is hydrogen or a (C₁-C₆)alkyl, and R_{y2} is (C₁-C₆)alkyl. Examples of such drying agents are triethyl orthoacetate, CH₃C(OCH₂CH₃)₃; trimethyl orthoacetate, CH₃C(OCH₃)₃; triethyl orthoformate, HC(OCH₂CH₃)₃; and trimethyl orthoformate, HC(OCH₃)₃. Additionally, molecular sieves, magnesium sulfate, calcium chloride, and sodium sulfate may be used. Optionally, mixtures of drying agents may be used.

In embodiment one of Scheme Three

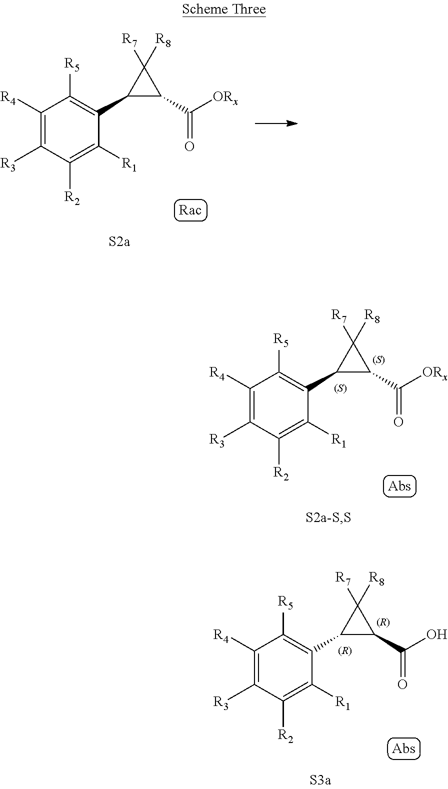

(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H;

(b) R₇ and R₈ are each independently F, Cl, Br, or I; and

(c) Rₓ is (C₁-C₆)alkyl or (C₁-C₆)hydroxyalkyl.

In embodiment two of Scheme Three, wherein R₂ and R₄ are CF₃; R₁, R₃, and R₅ H; R₇ and R₈ are Cl; and Rₓ is CH₃, this molecule is shown above in Scheme 2 and is named as trans-rac-methyl 3-(3,5-bis(trifluoromethyl)phenyl)-2,2-dichlorocyclopropane-1-carboxylate (also known as S2a-1).

In embodiment three of Scheme Three R₂ and R₄ are CF₃; R₁, R₃, and R₅ are H; and R₇ and R₈ are Cl. This molecule (“S3a-1”) is represented as follows.

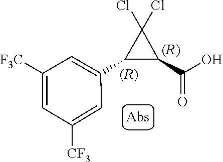

### (1R,3R)-3-(3,5-bis(trifluoromethyl)phenyl)-2,2-dichlorocyclopropane-1-carboxylic acid

The transformation in Scheme Three can be a chiral kinetic resolution, which involves using a chiral catalyst or reagent to promote selective transformation of one enantiomer (in this case the (R,R)-enantiomer of S2a) over the other ((S,S)-enantiomer of S2a)), giving a mixture of enantioenriched starting material and product, which can be separated by chemical and/or physical methods. The theoretical yield for similar kinetic resolutions is about 50%, as a racemic mixture (in this case S2a) is comprised of equal amounts of two enantiomers. Reference: Keith, J. M.; Larrow, J. F.; Jacobsen, E. N. Adv. Synth. Catal. 2001, 343, 5. In embodiment two of Scheme Three, two major components, enantioenriched portion of (S,S)-ester and (R,R)-acid product, are produced from this transformation; minor amounts of an (S,S)-acid and (R,R)-ester may also be present. After this transformation, the (R,R)-acid can be recovered using conventional separation and isolation techniques.

The transformation in Scheme Three is conducted in the presence of a hydrolase, preferably hydrolases that are classified under Enzyme Commission number EC3 “Hydrolases”; more preferably hydrolases that are classified under Enzyme Commission number EC3.1—acting on ester bonds; and most preferably hydrolases that are classified under Enzyme Commission number EC3.1.1—carboxylic ester hydrolases. Suitable hydrolyses will hydrolyze the ester in a racemic mixture of S2a to an enantiomerically enriched mixture of S3a. It is preferred if the production value (PV, as calculated in Equation 1) of S2a is greater than 20%, more preferably more than 30%, and most preferably more than 40%. Preferably, the enantiomeric excess (ee, as calculated in Equation 2) of S3a, where the (R,R)-enantiomer is greater than 80%, preferably, greater than 90%, more preferably greater than 95%, and most preferably greater than 99%. Suitable carboxylic ester hydrolases to use may be selected from Pseudomonas stutzeri lipase, Pseudomonas cepacia lipase, Alcaligenes sp. Lipase E, Alcaligenes sp. Lipase C, Pseudomonas fluorencens lipase, Burkholderia cepacia Lipase A, and Burkholderia cepacia Lipase B. Other enzymes may be used but it is desired that homology of these enzymes be at least 90% homologous, preferably at least 95% homologous with at least one of the above-mentioned carboxylic ester hydrolases. The amount of hydrolase, esterase, and lipase used may be from about 0.01% to about 200% by weight with respect to S2a, preferably about 0.1 to 1% by weight with respect to S2a. Commercially, hydrolases are available from a variety of suppliers, for example Almac Group Limited; Amano Enzyme USA Co., Ltd.; c-LEcta GmbH; Creative Enzymes; Codexis Inc.; Enzymaster (Ningbo) Bio-Engineering Co., Ltd.; Meito Sangyo Co., Ltd., Novozymes A/S. For further information, see Enzyme Catalysis in Organic Synthesis, 3 Vohlme Set edited by Karlheinz Drauz, Harald Gröger, and Oliver May, Chapter 46 (for example, see pages 1852-1854), by David Rozzell; and “Tabular Survey of Available Enzymes” p 1849-1985, copyrighted 2012 by Wiley-VCh Verlag GmbH & Co. KGaA, and published 2012 Wiley-VCh Verlag GmbH & Co. KGaA. Optionally, a hydrolase can be immobilized or supported on a polymer, silica, or other support material to allow for the recovery of the enzyme for further use. The following sequences or those with 90% or greater amino acid sequence homology may be used for this transformation AND61386 (SEQ ID NO:1); AAA50466 (UniProt P22088) (SEQ ID NO:2); AAC60400 (UniProt P25275) (SEQ ID NO:3); EGD05490 (UniProt FOFZ41) (SEQ ID NO:4); CAA83122 (SEQ ID NO:5); BBC47796 (SEQ ID NO:6); UniProt P20261(SEQ ID NO:7).

The transformation of Scheme Three is conducted in the presence of water (H₂O), preferably deionized water.

The transformation of Scheme Three is conducted optionally in the presence of an aqueous buffer where the hydrolase is preferably soluble. Examples of suitable buffers are sodium phosphate, 2-[bis(2-hydroxyethyl)amino]-2-(hydroxymethyl)propane-1,3-diol (“Bis-tris methane”), 2-[4-(2-hydroxyethyl)piperazin-1-yl]ethanesulfonic acid (“HEPES”), potassium phosphate, 3-morpholinopropane-1-sulfonic add (“MOPS”), piperazine-N,N′-bis(2-ethanesulfonic acid) (“PIPES”), sodium citrate, 3-{[1,3-dihydroxy-2-(hydroxymethyl)propan-2-yl]amino}propane-1-sulfonic acid (“TAPS”), lysine, 2-amino-2-(hydroxymethyl)propane-1,3-diol (“TRIS”), and mixtures thereof. The buffer concentration may be about 0.0001 molar (M) to about 0.5 M, preferably about 0.05 M to about 0.2 M.

The transformation of Scheme Three may be conducted optionally in the presence of an amine base. Examples of amine bases are lysine, ethanolamine, glycine, and mixtures thereof. Consequently, lysine may be used both as a buffer. The amount of amine base is from about 0.1 wt % to about 150 wt %, preferably about 10 wt % to about 40 wt % based on the weight of S2a.

The transformation of Scheme Three may be conducted in the presence of a co-solvent. Examples of co-solvents are acetone, acetonitrile, methyl tetrahydrofuran, methyl tert-butyl ether, hexane, toluene, methyl ethyl ketone, cyclopentyl methyl ether, dimethyl sulfoxide, dimethoxyethane, and mixtures thereof. A co-solvent may be useful to increase the solubility of S2a. The amount of co-solvent used may be from about 1% by volume to about 90% of the entire volume, preferably about 10% by volume to about 40% by volume based on the entire volume.

The transformation in Scheme Three may be conducted at ambient temperatures and pressures. However, higher or lower temperatures and pressures may be used. Currently, temperatures from about 0° C. to about 80° C. may be used; preferably temperatures from about 15° C. to about 60° C. may be used; more preferably temperatures from about 25° C. to about 50° C. may be used. Currently, pressures from about 10 kPa to about 1000 kPa may be used; preferably pressures from about 50 kPa to about 150 kPa may be used. The pH of the transformation mixture should be in the range of about pH 5 to about pH 11, preferably about pH 6 to about pH 10.

### EXAMPLES

These examples are for illustration purposes and are not to be construed as limiting this disclosure to only the embodiments disclosed in these examples.

Starting materials, reagents, and solvents that were obtained from commercial sources were used without further purification. Anhydrous solvents were purchased as Sure/Seal™ from Aldrich and were used as received. Melting points were obtained on a Thomas Hoover Unimelt capillary melting point apparatus or an OptiMelt Automated Melting Point System from Stanford Research Systems and are uncorrected. Examples using “room temperature” were conducted in climate controlled laboratories with temperatures ranging from about 20° C. to about 24° C. Molecules are given their known names, named according to naming programs within ISIS Draw, ChemDraw, or ACD Name Pro. If such programs are unable to name a molecule, such molecule is named using conventional naming rules. ¹H NMR spectral data are in ppm (δ) and were recorded at 300, 400, 500, or 600 MHz; ¹³C NMR spectral data are in ppm (δ) and were recorded at 75, 100, or 150 MHz; and ¹⁹F NMR spectral data are in ppm (δ) and were recorded at 376 MHz, unless otherwise stated.

### Example 1a

### Synthesis of (E)-1-(3,3-diethoxyprop-1-en-1-yl)-3,5-bis(trifluoromethyl)benzene

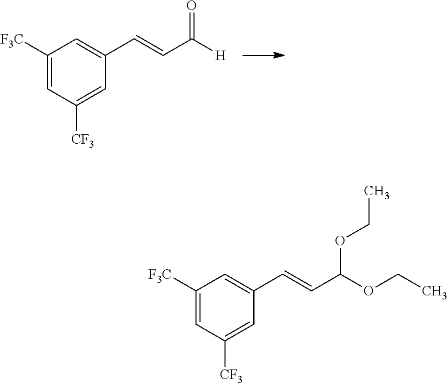 [SMILES: O=C/C=C/c1cc(C(F)(F)F)cc(C(F)(F)F)c1 ; CCOC(/C=C/c1cc(C(F)(F)F)cc(C(F)(F)F)c1)OCC]

In a 3-liter (L) single-neck flask equipped with a stir bar: (E)-3-(3,5-bis(trifluoromethyl)phenyl)acrylaldehyde (290 grams (g), 1081 millimoles (mmol)) was stirred in ethanol (360 milliliters (mL)). Triethyl orthoformate (187 mL, 1125 mmol) was added followed by pyridin-1-ium 4-methylbenzenesulfonate (PPTS; 0.544 g, 2.163 mmol). The suspension was stirred at 20° C. After 4 hours, 4-methylmorpholine (2.378 mL, 21.63 mmol) was added to quench the acid. The mixture was concentrated via rotary evaporation (45° C.) to remove ethanol. The concentrate was partitioned between heptane and water (pH 7). The organic portion was separated, dried over sodium sulfate, and concentrated (45° C., <1 Torr) to provide a yellow oil (341 g, 93%): ¹H NMR (300 MHz, Chloroform-d) δ 7.82 (s, 2H), 7.76 (s, 1H), 6.80 (dd, J=16.2, 1.2 Hz, 1H), 6.36 (dd, J=16.1, 4.6 Hz, 1H), 5.12 (dd, J=4.6, 1.3 Hz, 1H), 3.87-3.35 (m, 4H), 1.27 (t, J=7.1 Hz, 6H); ¹⁹F NMR (376 MHz, Chloroform-d) δ −63.05.

In accordance with the procedure disclosed and exemplified in example 1a, the following molecule was prepared.

### (E)-4-(3,3-diethoxyprop-1-en-1-yl)-1-fluoro-2-(trifluoromethyl)benzene

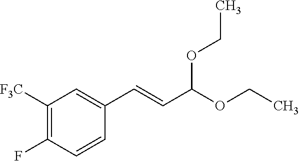 [SMILES: CCOC(/C=C/c1ccc(F)c(C(F)(F)F)c1)OCC]

¹H NMR (400 MHz, Chloroform-d) δ 7.62 (dd, J=6.9, 2.3 Hz, 1H), 7.56 (ddd, J=7.5, 4.7, 2.3 Hz, 1H), 7.15 (t, J=9.3 Hz, 1H), 6.69 (d, J=16.1 Hz, 1H), 6.19 (dd, J=16.1, 4.9 Hz, 1H), 5.07 (dd, J=4.9, 1.2 Hz, 1H), 3.71 (dq, J=9.5, 7.1 Hz, 2H), 3.56 (dq, J=9.4, 7.1 Hz, 2H), 1.26 (t, J=7.1 Hz, 6H); GC-MS m/z 292.1.

### Example 1b

### Synthesis of trans-rac-1-2,2-dichloro-3-(diethoxymethyl)cyclopropyl)-3,5-bis(trifluoromethyl)benzene (S1a-1)

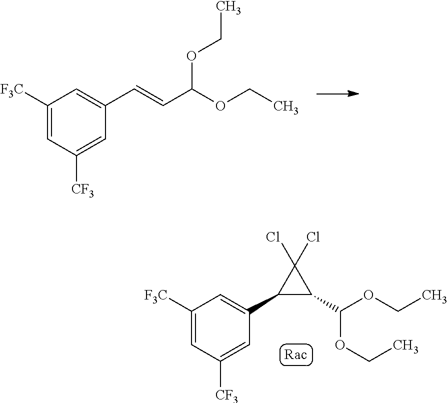 [SMILES: CCOC(/C=C/c1cc(C(F)(F)F)cc(C(F)(F)F)c1)OCC ; CCOC(OCC)[C@@H]1[C@@H](c2cc(C(F)(F)F)cc(C(F)(F)F)c2)C1(Cl)Cl]

In a 5-L jacketed reactor equipped with an overhead stirrer, baffles, condenser, temperature probe, and nitrogen inlet: (E)-1-(3,3-diethoxyprop-1-en-1-yl)-3,5-bis(trifluoromethyl)benzene (125 g, 365 mmol) and benzyltriethylammonium chloride (0.412 g, 1.826 mmol) were stirred in a mixture of chloroform (1180 mL) and heptane (535 mL). 50% Sodium hydroxide (NaOH, aqueous; 484 mL, 9130 mmol) was added via addition funnel over 30 minutes. The jacket temperature was set to 45° C. The reaction mixture was stirred vigorously at 45° C. until the reaction reached 90 to 95% conversion. After the reaction mixture was cooled to 20° c, water (1.2 L) was charged to the reactor, and the mixture was stirred for 5 minutes. After the layers separated, the aqueous layer was removed, and the organic layer was washed with additional water (1.2 L). The organic layer was concentrated under vacuum to obtain a brown oil containing the desired product S1a-1 (85%, in-pot): ¹H NMR (400 MHz, Chloroform-d) δ 7.83 (s, 1H), 7.71 (s, 2H), 4.64 (d, J=6.1 Hz, 1H), 3.82-3.55 (m, 4H), 2.94 (d, J=8.4 Hz, 1H), 2.35 (dd, J=8.5, 6.1 Hz, 1H), 1.32 (t, J=7.0 Hz, 3H), 1.21 (t, J=7.1 Hz, 3H); ¹⁹F NMR (471 MHz, CDCl₃) δ −62.87.

In accordance with the procedure disclosed and exemplified in example 1b, the following molecule was prepared.

### trans-rac-4-(2,2-dichloro-3-(diethoxymethyl)cyclopropyl)-1-fluoro-2-(trifluoromethyl)benzene (S1a-2)

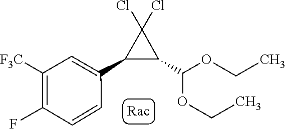 [SMILES: CCOC(OCC)[C@@H]1[C@@H](c2ccc(F)c(C(F)(F)F)c2)C1(Cl)Cl]

¹H NMR (400 MHz, Chloroform-d) δ 7.50 (dd, J=6.7, 2.3 Hz, 1H), 7.43 (ddd, J=7.4, 4.5, 2.4 Hz, 1H), 7.19 (t, J=9.3 Hz, 1H), 4.60 (d, J=6.2 Hz, 1H), 3.83-3.56 (m, 4H), 2.83 (d, J=8.4 Hz, 1H), 2.26 (dd, J=8.4, 6.2 Hz, 1H), 1.31 (t, J=7.0 Hz, 3H), 1.20 (t, J=7.1 Hz, 3H); GC-MS m/z 375.1.

### Example 1c

### Synthesis of trans-rac-3-(3,5-bis(trifluoromethyl)phenyl)-2,2-dichlorocyclopropane-1-carboxylic acid (S1b-1)

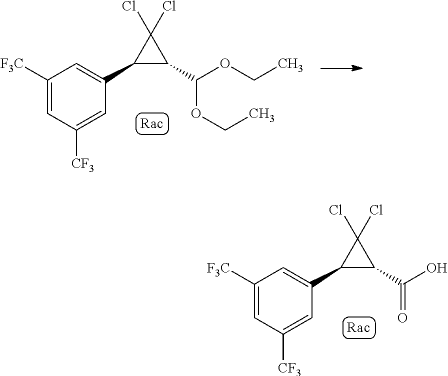 [SMILES: CCOC(OCC)[C@@H]1[C@@H](c2cc(C(F)(F)F)cc(C(F)(F)F)c2)C1(Cl)Cl ; O=C(O)[C@@H]1[C@@H](c2cc(C(F)(F)F)cc(C(F)(F)F)c2)C1(Cl)Cl]

In a 3-L four-neck flask equipped with a mechanical stirrer, temperature probe, and condenser: trans-rac-1-(2,2-dichloro-3-(diethoxymethyl)cyclopropyl)-3,5-bis(trifluoromethyl)benzene (S1a-1; 96.5 g, 227 mmol) was stirred in acetic acid (779 mL). Water (82 mL) was added. OXONE® (140 g, 227 mmol) was added. The mixture was heated to 30° C. After 30 minutes, the temperature was slowly increased to 45° C. After 20 hours, the mixture was cooled to 25° C. Sodium bisulfite (23.6 g, 227 mmol) was added to quench remaining peroxides. After stirring for 30 minutes, no peroxides were present when tested with potassium iodide (KI) paper. Acetonitrile (1.5 L) was added. The mixture was filtered, and the filtrate was concentrated. The concentrate was partitioned between ethyl acetate (500 mL) and brine (200 mL). The ethyl acetate portion was concentrated to provide an orange oil. The oil was partitioned between acetonitrile (500 mL) and heptane (300 mL). The heptane portion was extracted with acetonitrile (100 mL). The acetonitrile portion was concentrated to provide product S1b-1 as an orange oil (81.8 g, 93%): ¹H NMR (400 MHz, Chloroform-d) δ 7.89 (s, 1H), 7.73 (s, 2H), 3.60 (d, J=8.2 Hz, 1H), 2.99 (d, J=8.3 Hz, 1H); ¹⁹F NMR (471 MHz, Chloroform-d) δ −62.90.

### Example 1d

### Synthesis of trans-rac-3-(3,5-bis(trifluoromethyl)phenyl)-2,2-dichlorocyclopropane-1-carboxylic acid (S1b-1)

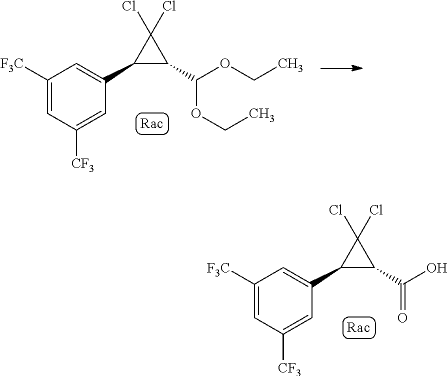 [SMILES: CCOC(OCC)[C@@H]1[C@@H](c2cc(C(F)(F)F)cc(C(F)(F)F)c2)C1(Cl)Cl ; O=C(O)[C@@H]1[C@@H](c2cc(C(F)(F)F)cc(C(F)(F)F)c2)C1(Cl)Cl]

In a 1-L jacketed reactor equipped with a mechanical stirrer, temperature probe, condenser, and nitrogen inlet: trans-rac-1-(2,2-dichloro-3-(diethoxymethyl)cyclopropyl)-3,5-bis(trifluoromethyl)benzene (S1a-1; 127 g, 299 mmol) was stirred in acetonitrile (478 mL). Water (118 mL) was added. OXONE® (184 g, 299 mmol) was added. The reaction mixture was heated to 35° C. After 24 hours, analysis indicated complete conversion. The reaction mixture was cooled to 20° C. The mixture tested positive for peroxides. Sodium bisulfite (10.88 g, 105 mmol) was added in small portions until the mixture tested negative for peroxides. Ethyl acetate (1.5 L) was added. The organic portion was separated and concentrated to provide a yellow oil. The oil was taken up in acetonitrile (600 mL) and partitioned with n-heptane (2×200 mL). The acetonitrile layer was concentrated. The acetonitrile portion was concentrated to dryness and the residue was stirred up in n-heptane (800 mL) and stirred overnight. The mixture was cooled in an ice bath. After 2 hours, the slurry was vacuum filtered, and a solid was collected and vacuum-dried to give S1b-1 (102.6 g, 95%).

### Example 1e

### Synthesis of trans-rac-2,2-dichloro-3-(4-fluoro-3-(trifluoromethyl)phenyl)cyclopropane-1-carboxylic acid (S1b-2)

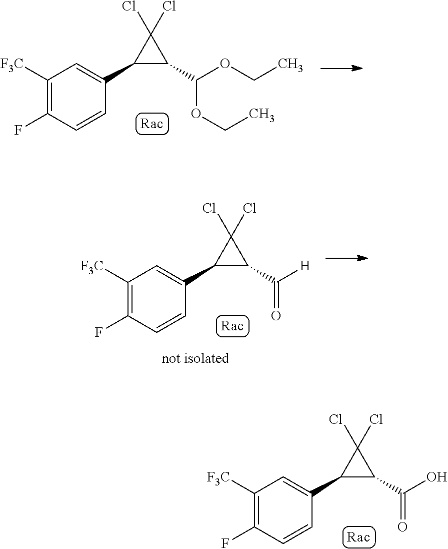 [SMILES: CCOC(OCC)[C@@H]1[C@@H](c2ccc(F)c(C(F)(F)F)c2)C1(Cl)Cl ; O=C[C@@H]1[C@@H](c2ccc(F)c(C(F)(F)F)c2)C1(Cl)Cl ; O=C(O)[C@@H]1[C@@H](c2ccc(F)c(C(F)(F)F)c2)C1(Cl)Cl]

In a 1-L four-neck flask equipped with a temperature probe, condenser, and nitrogen inlet: trans-rac-4-(2,2-dichloro-3-(diethoxymethyl)cyclopropyl)-1-fluoro-2-(trifluoromethyl)benzene (S1a-2; 30 g, 80 mmol) was stirred in acetic acid (276 mL). Water (27.6 mL) was added. The mixture was heated to 40° C. After 16 hours, ¹H-NMR analysis showed complete formation of trans-rac-2,2-dichloro-3-(4-fluoro-3-(trifluoromethyl)phenyl)cyclopropane-1-carbaldehyde (>99% conversion): ¹H NMR (400 MHz, Chloroform-d) δ 9.55 (d, J=4.0 Hz, 1H), 7.50 (dd, J=6.6, 2.3 Hz, 1H), 7.50-7.42 (m, 1H), 7.24 (t, J=9.1 Hz, 1H), 3.57 (d, J=7.9 Hz, 1H), 2.93 (dd, J=8.0, 4.0 Hz, 1H); ¹⁹F NMR (376 MHz, Chloroform-d) δ −61.51 (d, J=12.9 Hz), −113.97 (q, J=12.4 Hz).

As such, OXONE® (30.4 g, 49.4 mmol) was added to the reaction mixture. The mixture was heated to 50° C. and stirred for 20 hours. The mixture was allowed to cool to 23° C. Sodium bisulfite (3.95 g, 38 mmol) was added portion-wise to quench remaining peroxides. The mixture was diluted with acetonitrile (500 mL). After stirring for 1 hour, the mixture was filtered and concentrated. The concentrate was partitioned between ethyl acetate and water. The organic portion was dried and concentrated to provide S1b-2 as an orange oil (24 g, 95%): ¹H NMR (300 MHz, Chloroform-d) δ 7.53-7.42 (m, 2H), 7.23 (t, J=9.2 Hz, 1H), 3.48 (d, J=8.3 Hz, 1H), 2.88 (d, J=8.3 Hz, 1H); ¹⁹F NMR (376 MHz, Chloroform-d) δ −61.49 (d, J=12.9 Hz), −114.27 (q, J=12.7 Hz).

### Example 1f

### Synthesis of trans-rac-3-(3,5-bis(trifluoromethyl)phenyl)-2,2-dichlorocyclopropane-1-carboxylic acid (S1b-1)

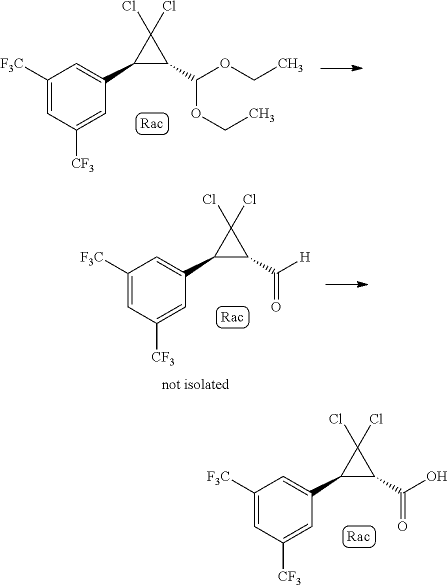 [SMILES: CCOC(OCC)[C@@H]1[C@@H](c2cc(C(F)(F)F)cc(C(F)(F)F)c2)C1(Cl)Cl ; O=C[C@@H]1[C@@H](c2cc(C(F)(F)F)cc(C(F)(F)F)c2)C1(Cl)Cl ; O=C(O)[C@@H]1[C@@H](c2cc(C(F)(F)F)cc(C(F)(F)F)c2)C1(Cl)Cl]

To a 100-mL reactor equipped with a temperature probe and nitrogen inlet were added acetic acid (280 mL) and water (28.0 mL). The reaction mixture was heated to 50° C. trans-rac-1-(2,2-Dichloro-3-(diethoxymethyl)cyclopropyl)-3,5-bis(trifluoromethyl)benzene (S1a-1; 30 g, 70.6 mmol) was added in one portion. The reaction progress was monitored by ¹H-NMR and GC-MS analysis. After 60 minutes, complete conversion of starting material to trans-rac-3-(3,5-bis(trifluoromethyl)phenyl)-2,2-dichlorocyclopropane-1-carbaldehyde was observed (>99% conversion): ¹H NMR (300 MHz, Chloroform-d) δ 9.61 (d, J=3.7 Hz, 1H), 7.92-7.85 (m, 1H), 7.78-7.67 (m, 2H), 3.67 (d, J=8.0 Hz, 1H), 3.05 (dd, J=8.0, 3.7 Hz, 1H); ¹⁹F NMR (376 MHz, Chloroform-d) δ −62.92; GC-MS m/z 315.

As such, OXONE® (43.4 g, 70.6 mmol) was added in one portion to the reaction mixture. A mild exotherm was observed. After 3 hours, ¹H NMR analysis showed complete conversion to desired carboxylic acid product. The mixture was cooled to 20° C. and stirred overnight. The mixture was cooled to 5° C. and quenched with sodium bisulfite (9.54 g, 92 mmol) in water (30 mL). The mixture was warmed to 25° C. and diluted with water (200 mL). Methyl tert-butyl ether (MTBE; 300 mL) was added. The aqueous layer was extracted with MTBE (300 mL). The combined organic extracts were washed with water (3×200 mL), collected, dried (MgSO₄), filtered and concentrated. The residue was diluted with heptane (200 mL) and concentrated to dryness. A seed of solid racemic product was added. Solids began to form. The solids were triturated with n-heptane (200 mL), filtered and washed with n-heptane to afford desired product (23.5 g, 91%).

### Example 1g

### Synthesis of trans-rac-3-(3,5-bis(trifluoromethyl)phenyl)-2,2-dichlorocyclopropane-1-carboxylic acid (S1b-1)

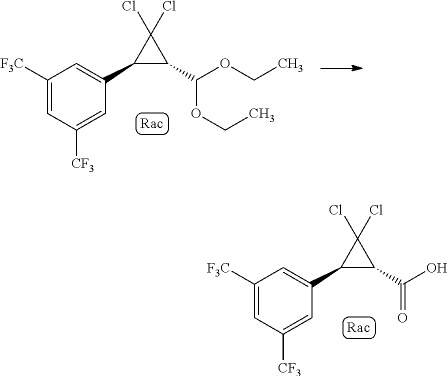 [SMILES: CCOC(OCC)[C@@H]1[C@@H](c2cc(C(F)(F)F)cc(C(F)(F)F)c2)C1(Cl)Cl ; O=C(O)[C@@H]1[C@@H](c2cc(C(F)(F)F)cc(C(F)(F)F)c2)C1(Cl)Cl]

In a jacketed reactor equipped with a temperature probe, nitrogen inlet, and overhead stirrer: trans-rac-1-(2,2-dichloro-3-(diethoxymethyl)cyclopropyl)-3,5-bis(trifluoromethyl)benzene (S1a-1; 127 g, 78.6 wt % pure, 235 mmol) was dissolved in acetic acid (538 mL, 9391 mmol). Sulfuric acid (0.63 mL, 11.7 mmol) was added. The mixture was warmed to 50° C. Hydrogen peroxide (30 wt %; 48 mL, 470 mmol) was added over 8 hours. After the complete addition of hydrogen peroxide, the mixture was held at 50° C. for 14 hours. Sodium bisulfite (29.3 g, 282 mmol) was added as a 10 wt % solution in water. After 30 minutes, the reaction mixture tested negative for residual peroxides with potassium iodide (KI) starch paper. The mixture was distilled (15-38 torr, 60-70° C.) until 606.8 g distillate was removed. Heptanes (743 mL) was added to the residue at 60-70° C. and the layers were separated. The heptane layer was washed twice with water (127 mL). The heptanes layer was concentrated by distillation until 255 g of distillate was collected. The concentrate was cooled to −5° C. and was held for 60 minutes. The resultant solid was collected by vacuum filtration and was vacuum dried to afford S1b-1 (71.7 g, 82%).

### Example 1h

### Synthesis of trans-rac-2,2-dichloro-3-(3-chloro-4-fluorophenyl)cyclopropane-1-carboxylic acid (S1b-3)

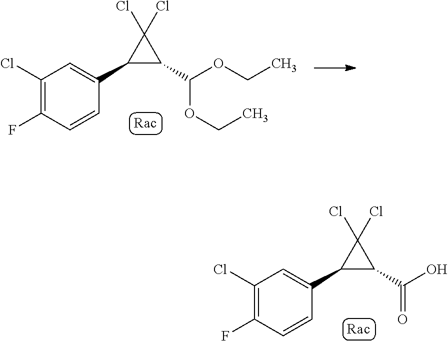 [SMILES: CCOC(OCC)[C@@H]1[C@@H](c2ccc(F)c(Cl)c2)C1(Cl)Cl ; O=C(O)[C@@H]1[C@@H](c2ccc(F)c(Cl)c2)C1(Cl)Cl]

In a 100 mL single-neck flask equipped with a stir bar: trans-rac-2-chloro-4-(2,2-dichloro-3-(diethoxymethyl)cyclopropyl)-1-fluorobenzene (prepared as in Heemstra et al., WO 2016168059 A1; 4.246 g, 12.43 mmol) was stirred in acetic acid (28.5 mL). Sulfuric acid (0.61 g, 0.621 mmol) was added. The mixture was heated to 50° C. Hydrogen peroxide (30 wt %, 2.54 mL, 24.86 mmol) was added over 8 hours. After 22 hours, the mixture was cooled to 25° C. Sodium bisulfite (1.552 g, 14.91 mmol) was added as a 10 wt % solution in water to quench remaining peroxides. The mixture was concentrated by approximately one-half volume under vacuum. Water (14 mL) was added. The mixture was extracted three times with dichloromethane (10 mL). The combined extracts were concentrated under vacuum to afford S1b-3 (3.18 g, 90%): ¹H NMR (400 MHz, Chloroform-d) δ 7.39-7.34 (m, 1H), 7.20-7.15 (m, 2H), 3.43 (d, J=8.2 Hz, 1H), 2.88 (d, J=8.3 Hz, 1H); ¹³C NMR (126 MHz, Chloroform-d) δ 158.16 (d, J=250.6 Hz), 129.40 (d, J=4.0 Hz), 128.69 (d, J=7.4 Hz), 121.57 (d, J=18.2 Hz), 116.99 (d, J=21.5 Hz); ¹⁹F NMR (471 MHz, Chloroform-d) δ −115.20.

### Example 2a

### Synthesis of trans-rac-methyl 3-(3,5-bis(trifluoromethyl)phenyl)-2,2-dichlorocyclopropane-1-carboxylate (S2a-1)

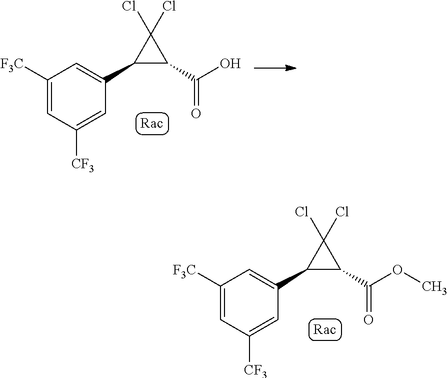 [SMILES: O=C(O)[C@@H]1[C@@H](c2cc(C(F)(F)F)cc(C(F)(F)F)c2)C1(Cl)Cl ; COC(=O)[C@@H]1[C@@H](c2cc(C(F)(F)F)cc(C(F)(F)F)c2)C1(Cl)Cl]

In a 3-L single-neck flask equipped with a mechanical stirrer, temperature probe, and condenser: trans-rac-3-(3,5-bis(trifluoromethyl)phenyl)-2,2-dichlorocyclopropane-1-carboxylic acid (S1b-1; 123.6 g, 337 mmol) was stirred in methanol (1362 mL, 33700 mmol). Sulfuric acid (1.077 mL, 20.20 mmol) was added. The mixture was heated to a gentle reflux (about 65° C.). After 20 hours, the mixture was allowed to cool. 4-Methylmorpholine (3.33 mL, 30.3 mmol) was added. The mixture was concentrated. The concentrate was taken up in hexanes (1.5 L) and washed with water (500 mL). The hexanes portion was dried and concentrated to provide S2a-1 as a solid (123.72 g, 92%): ¹H NMR (300 MHz, Chloroform-d) δ 7.87 (s, 1H), 7.71 (s, 2H), 3.88 (s, 3H), 3.58 (d, J=8.3 Hz, 1H), 2.95 (d, J=8.3 Hz, 1H); ¹⁹F NMR (376 MHz, Chloroform-d) δ −62.93.

In accordance with the procedures disclosed in Scheme 2 and exemplified in example 2a, the following molecules were prepared.

### trans-rac-methyl 2,2-dichloro-3-(4-fluoro-3-(trifluoromethyl)phenyl)cyclopropane-1-carboxylate (S2a-2)

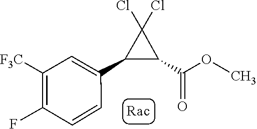 [SMILES: COC(=O)[C@@H]1[C@@H](c2ccc(F)c(C(F)(F)F)c2)C1(Cl)Cl]

¹H NMR (400 MHz, Chloroform-d) δ 7.52-7.41 (m, 2H), 7.22 (t, J=9.2 Hz, 1H), 3.86 (s, 3H), 3.48 (d, J=7.4 Hz, 1H), 2.85 (d, J=8.3 Hz, 1H); ¹⁹F NMR (376 MHz, Chloroform-d) δ −61.48 (d, J=12.8 Hz), −114.60 (q, J=12.5 Hz).

### trans-rac-methyl 2,2-dichloro-3-(3,4-dichlorophenyl)cyclopropane-1-carboxylate (S2a-3)

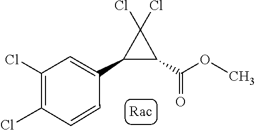 [SMILES: COC(=O)[C@@H]1[C@@H](c2ccc(Cl)c(Cl)c2)C1(Cl)Cl]

¹H NMR (400 MHz, Chloroform-d) δ 7.45 (d, J=8.3 Hz, 1H), 7.35 (dd, J=2.1, 0.7 Hz, 1H), 7.11 (ddd, J=8.3, 2.1, 0.7 Hz, 1H), 3.85 (s, 3H), 3.42 (d, J=8.3 Hz, 1H), 2.82 (d, J=8.3 Hz, 1H).

### Example 2b

### Synthesis of trans-rac-methyl 3-(3,5-bis(trifluoromethyl)phenyl)-2,2-dichlorocyclopropane-1-carboxylate (S2a-1)

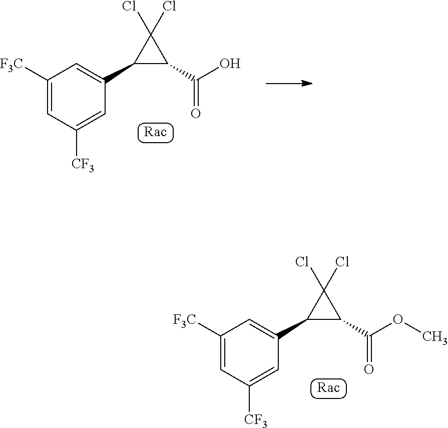 [SMILES: O=C(O)[C@@H]1[C@@H](c2cc(C(F)(F)F)cc(C(F)(F)F)c2)C1(Cl)Cl ; COC(=O)[C@@H]1[C@@H](c2cc(C(F)(F)F)cc(C(F)(F)F)c2)C1(Cl)Cl]

In a glass vial equipped with a magnetic stir bar: trans-rac-3-(3,5-bis(trifluoromethyl)phenyl)-2,2-dichlorocyclopropane-1-carboxylic acid (S1b-1; 1.0 g, 2.72 mmol) and trimethyl orthoformate (0.343 mL, 3.13 mmol) were added. A solution of sulfuric acid (5.45 mg, 0.054 mmol) in methanol (0.441 mL, 10.90 mmol) was added in one portion. The reaction mixture was heated to 65° C. and stirred for 19 hours. The temperature was increased to 75° C. to distill off volatiles for 1 hour. The residue was cooled to 50-55° C. then heptane (4.0 mL) and potassium carbonate (0.015 g, 0.11 mmol) in water (2.0 mL) were added. The mixture was stirred for 15 minutes and then the layers were separated. The heptane layer was concentrated under vacuum to afford S2a-1 (1.0 g, 96%).

In accordance with the procedures disclosed in Scheme 2 and exemplified in example 2b, the following molecules were prepared

### trans-rac-methyl 2,2-dichloro-3-(3,5-dichlorophenyl)cyclopropane-1-carboxylate (S2a-4)

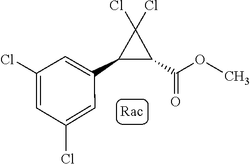 [SMILES: COC(=O)[C@@H]1[C@@H](c2cc(Cl)cc(Cl)c2)C1(Cl)Cl]

¹H NMR (500 MHz, Chloroform-d) δ 7.35-7.33 (m, 1H), 7.17-7.14 (m, 2H), 3.85 (s, 3H), 3.41 (d, J=8.3 Hz, 1H), 2.84 (d, J=8.3 Hz, 1H); ¹³C NMR (126 MHz, Chloroform-d) δ 166.62, 135.94, 135.32, 128.63, 127.47, 61.30, 53.21, 39.37, 37.46.

### Example 2c

### Synthesis of trans-rac-methyl 3-(3,5-bis(trifluoromethyl)phenyl)-2,2-dichlorocyclopropane-1-carboxylate (S2a-1)

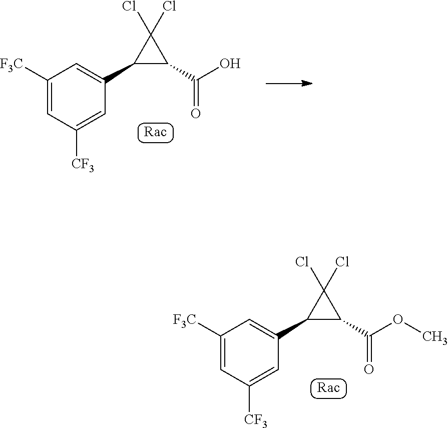 [SMILES: O=C(O)[C@@H]1[C@@H](c2cc(C(F)(F)F)cc(C(F)(F)F)c2)C1(Cl)Cl ; COC(=O)[C@@H]1[C@@H](c2cc(C(F)(F)F)cc(C(F)(F)F)c2)C1(Cl)Cl]

In a glass vial equipped with a magnetic stir bar: trans-rac-3-(3,5-bis(trifluoromethyl)phenyl)-2,2-dichlorocyclopropane-1-carboxylic acid (S1b-1; 1.0 g, 2.72 mmol) and trimethyl orthoformate (0.343 mL, 3.13 mmol) were added. A solution of sulfuric acid (5.45 mg, 0.054 mmol) in methanol (0.441 mL, 10.90 mmol) was added in one portion. The reaction mixture was heated to 65° C. and stirred for 19 hours. The temperature was increased to 75° C. to distill off volatiles for 1 hour. The residue was cooled to ambient temperature affording desired product S2a-1 (1.0 g, 96%). The residue was cooled to 50-55° C. then heptane (4.0 mL) and potassium carbonate (0.015 g, 0.11 mmol) in water (2.0 mL) were added. The mixture was stirred for 15 minutes and then the layers were separated. The heptane layer was concentrated under vacuum to afford S2a-1 (1.0 g, 96%). Optionally, the residue could be partitioned between heptane (4.0 mL) and potassium carbonate (0.015 g, 0.11 mmol) in water (2.0 mL) at 50-55° C. The heptane layer can be concentrated under vacuum to afford S2a-1.

### Example 2d

### Synthesis of trans-rac-methyl 3-(3,5-bis(trifluoromethyl)phenyl)-2,2-dichlorocyclopropane-1-carboxylate (S2a-1)

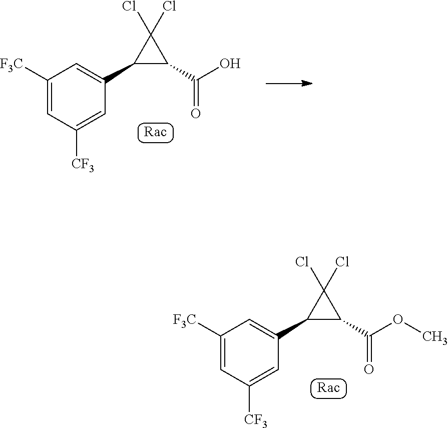 [SMILES: O=C(O)[C@@H]1[C@@H](c2cc(C(F)(F)F)cc(C(F)(F)F)c2)C1(Cl)Cl ; COC(=O)[C@@H]1[C@@H](c2cc(C(F)(F)F)cc(C(F)(F)F)c2)C1(Cl)Cl]

In a glass reactor equipped with a mechanical stirrer and reflux condenser: trans-rac-3-(3,5-bis(trifluoromethyl)phenyl)-2,2-dichlorocyclopropane-1-carboxylic acid (S1b-1; 93 wt %, 260 g, 659 mmol) and trimethyl orthoformate (80.4 g, 758 mmol) were added. Methanol (84.4 g, 2638 mmol) was added. Sulfuric acid (1.3 g, 13.3 mmol) was added. The reaction mixture was heated to 65° C. and stirred for 12 hours. The temperature was increased to 75° C. and volatiles were removed by distillation. The residue was cooled to 50-55° C. Heptanes (712.4 g) was added. A solution of potassium carbonate (3.64 g, 26.3 mmol) in water (520 g) was added. The mixture was stirred for 15-30 minutes. The layers were separated and the organic layer was concentrated by vacuum distillation affording desired product S2a-1 (259 g, 96 wt %, 96%).

### Example 2e

### Synthesis of trans-rac-methyl 3-(3,5-bis(trifluoromethyl)phenyl)-2,2-dichlorocyclopropane-1-carboxylate (S2a-5)

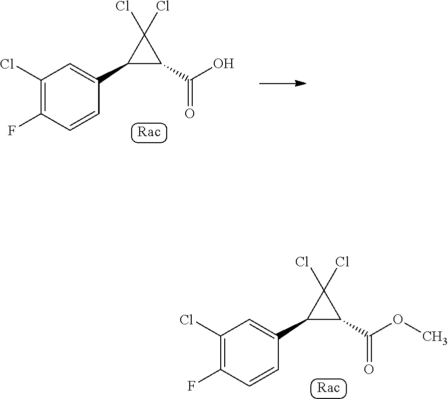 [SMILES: O=C(O)[C@@H]1[C@@H](c2ccc(F)c(Cl)c2)C1(Cl)Cl ; COC(=O)[C@@H]1[C@@H](c2ccc(F)c(Cl)c2)C1(Cl)Cl]

In a 100 mL single-neck flask equipped with a stir bar and reflux condenser: trans-rac-methyl 3-(3,5-bis(trifluoromethyl)phenyl)-2,2-dichlorocyclopropane-1-carboxylate (S1b-3; 5.93 g, 20.93 mmol) and trimethyl orthoformate (2.66 mL, 24.05 mmol) were added. A solution of sulfuric acid (41 mg, 0.42 mmol) in methanol (3.38 mL, 84 mmol) was added in one portion. The reaction mixture was heated to 65° C. and stirred for 19 hours. The temperature was increased to 75° C. to distill off volatiles for 1 hour. The residue was cooled to ambient temperature. The residue was partitioned between dichloromethane (20 mL) and potassium bicarbonate solution (1.16 wt %, 10 mL). The dichloromethane portion was separated and concentrated to provide desired product S2a-5 (5.90 g, 95%): ¹H NMR (400 MHz, Chloroform-d) δ 7.30 (dq, J=6.8, 1.3 Hz, 1H), 7.17-7.12 (m, 2H), 3.85 (s, 3H), 3.42 (d, J=8.3, 1.0 Hz, 1H), 2.81 (d, J=8.3 Hz, 1H).

### Example 2f

### Synthesis of trans-rac-ethyl 3-(3,5-bis(trifluoromethyl)phenyl)-2,2-dichlorocyclopropane-1-carboxylate (S2a-6)

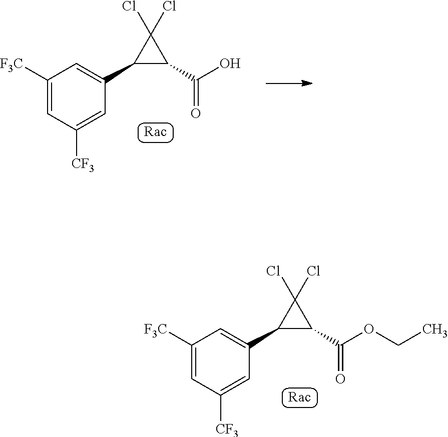 [SMILES: O=C(O)[C@@H]1[C@@H](c2cc(C(F)(F)F)cc(C(F)(F)F)c2)C1(Cl)Cl ; CCOC(=O)[C@@H]1[C@@H](c2cc(C(F)(F)F)cc(C(F)(F)F)c2)C1(Cl)Cl]

In a 30-mL vial equipped with a magnetic stir bar, reflux condenser, and nitrogen inlet: trans-rac-3-(3,5-bis(trifluoromethyl)phenyl)-2,2-dichlorocyclopropane-1-carboxylic acid (S1b-1; 3.0 g, 8.17 mmol) was mixed with triethyl orthoformate (1.56 mL, 9.40 mmol). A solution of sulfuric acid (16 mg, 0.163 mmol) in ethanol (1.91 mL) was added. The mixture was heated to 75° C. After stirring for 1 day, ethanol (1.91 mL) was added. After stirring for 3 days, the mixture was cooled to 50° C. and concentrated to an oil. The oil was partitioned between heptane (10 mL) and 10 wt % aqueous sodium bicarbonate solution (10 mL). The heptane layer was concentrated to afford the title compound S2a-6 as a yellow oil (2.56 g, 79%): ¹H NMR (400 MHz, Chloroform-d) δ 7.86 (s, 1H), 7.71 (d, J=1.6 Hz, 2H), 4.33 (qd, J=7.1, 1.9 Hz, 2H), 3.57 (d, J=8.3 Hz, 1H), 2.94 (d, J=8.3 Hz, 1H), 1.38 (t, J=7.2 Hz, 3H); ¹⁹F NMR (471 MHz, Chloroform-d) δ −62.91.

As an alternative to Schemes 1 and 2, the conversion of S1a to S2a can be conducted as illustrated in examples 2.5a and 2.5b.

### Example 2.5a

### Synthesis of trans-rac-ethyl 3-(3,5-bis(trifluoromethyl)phenyl)-2,2-dichlorocyclopropane-1-carboxylate (S2a-6)

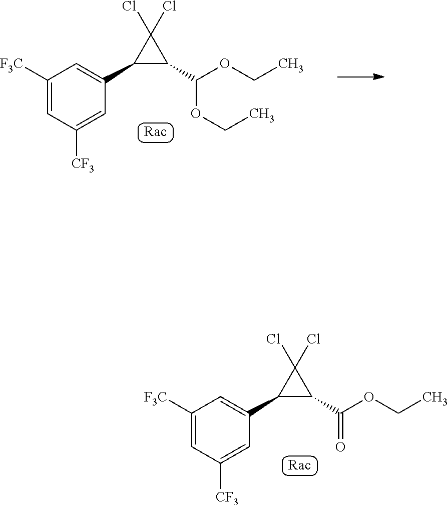 [SMILES: CCOC(OCC)[C@@H]1[C@@H](c2cc(C(F)(F)F)cc(C(F)(F)F)c2)C1(Cl)Cl ; CCOC(=O)[C@@H]1[C@@H](c2cc(C(F)(F)F)cc(C(F)(F)F)c2)C1(Cl)Cl]

In a 50-mL round bottom flask equipped with a magnetic stir bar, temperature probe, and nitrogen inlet: trans-rac-1-(2,2-dichloro-3-(diethoxymethyl)cyclopropyl)-3,5-bis(trifluoromethyl)benzene (S1a-1; 2.00 g, 4.70 mmol) was stirred in ethanol (9.39 mL). Water (0.25 mL, 14.11 mmol) was added. OXONE® (2.89 g, 4.70 mmol) was added. The reaction mixture was heated to 55° C. After 72 hours, analysis indicated complete conversion. The reaction mixture was cooled to 23° C. Sodium bisulfite (0.538 g, 5.17 mmol) was added. The mixture was stirred for 30 minutes. The mixture tested negative for peroxides with potassium iodide (KI) paper. The mixture was filtered. The filtrate was concentrated to an oil. The oil was partitioned between water (10 mL) and heptane (10 mL). The heptane layer was concentrated to afford the title compound S2a-6 as a yellow oil (1.384 g, 75%): ¹H NMR (400 MHz, Chloroform-d) δ 7.86 (s, 1H), 7.71 (d, J=1.6 Hz, 2H), 4.33 (qd, J=7.1, 1.9 Hz, 2H), 3.57 (d, J=8.3 Hz, 1H), 2.94 (d, J=8.3 Hz, 1H), 1.38 (t, J=7.2 Hz, 3H); ¹⁹F NMR (471 MHz, Chloroform-d) δ −62.91.

### Example 2.5b

### Synthesis of trans-rac-ethyl 3-(3,5-bis(trifluoromethyl)phenyl)-2,2-dichlorocyclopropane-1-carboxylate (S2a-6)

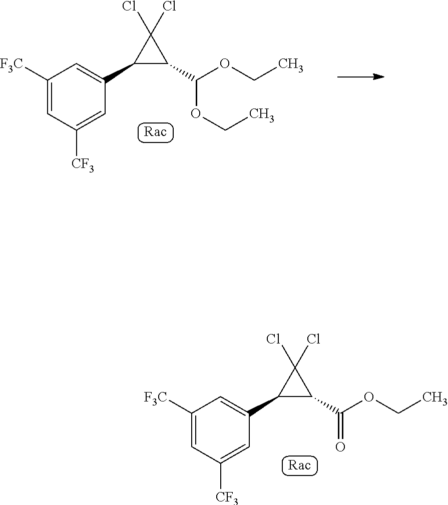 [SMILES: CCOC(OCC)[C@@H]1[C@@H](c2cc(C(F)(F)F)cc(C(F)(F)F)c2)C1(Cl)Cl ; CCOC(=O)[C@@H]1[C@@H](c2cc(C(F)(F)F)cc(C(F)(F)F)c2)C1(Cl)Cl]

In an 50-mL round bottom flask equipped with a magnetic stir bar, reflux condenser, and nitrogen inlet: trans-rac-1-(2,2-dichloro-3-(diethoxymethyl)cyclopropyl)-3,5-bis(trifluoromethyl)benzene (S1a-1; 1.00 g, 2.35 mmol) was stirred in ethanol (4.70 mL). Sodium percarbonate (0.369 g, 2.35 mmol) was added. Sulfuric acid (0.28 ml, 5.17 mmol) was added. The reaction mixture was heated to 70° C. After 20 hours, analysis indicated approximately 78% conversion. The reaction mixture was cooled to 23° C. Sodium bisulfite (0.073 g, 0.71 mmol) was added. The mixture was stirred for 20 minutes. The mixture tested negative for peroxides (KI paper). The mixture was diluted with heptane (10 ml) and the mixture was stirred for 5 minutes. The mixture was filtered. The solid cake was washed with heptane (5 ml). The filtrate was concentrated to an oil. The oil was dissolved in heptane (10 ml). The mixture was dried over sodium sulfate. The mixture was filtered and the filtrate was concentrated to afford the title compound S2a-6 as a yellow oil (65%, in pot) ¹H NMR (400 MHz, Chloroform-d) δ 7.86 (s, 1H), 7.71 (d, J=1.6 Hz, 2H), 4.33 (qd, J=7.1, 1.9 Hz, 2H), 3.57 (d, J=8.3 Hz, 1H), 2.94 (d, J=8.3 Hz, 1H), 1.38 (t, J=7.2 Hz, 3H); ¹⁹F NMR (471 MHz, Chloroform-d) δ −62.91.

### Example 3a

### Synthesis of (1R,3R)-3-(3,5-bis(trifluoromethyl)phenyl)-2,2-dichlorocyclopropane-1-carboxylic acid (S3a-1)

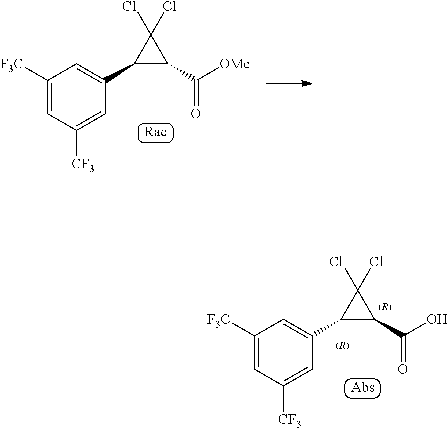

In a 5-L jacketed reactor vessel (18.4-centimeter (cm) diameter) equipped with a mechanical stirrer with a 9-cm diameter Rushton type impeller, two 1-cm wide baffles, a temperature probe, and a condenser, a solution of L-lysine monohydrate (60 g, 365 mmol) in deionized water (1800 mL) was charged. While stirring vigorously at 280 revolutions per minute (RPM), a solution of trans-rac-methyl 3-(3,5-bis(trifluoromethyl)phenyl)-2,2-dichlorocyclopropane-1-carboxylate (S2a-1); 300 g, 771 mmol) in acetone (1200 mL) was added to the reactor. The temperature of the vessel contents was increased to 30° C. and was mixed for 30 minutes. Pseudomonas stutzeri lipase (available from Almac Group Limited product code AH-04; 9 g) was added to the reaction as solid. The reaction mixture was stirred at 280 RPM and 30° C. for 24 hours when the reaction reached 45% production value of trans-rac-methyl 3-(3,5-bis(trifluoromethyl)phenyl)-2,2-dichlorocyclopropane-1-carboxylate (S2a-1) to the (1R,3R)-3-(3,5-bis(trifluoromethyl)phenyl)-2,2-dichlorocyclopropane-1-carboxylic acid (S3a-1).

Production value was calculated using Equation 1.

$$production value ( \% ) = 100 * 0.5 * ( 1 - \frac{Area units of ( R , R ) ester}{Area units of ( S , S ) ester} ) \qquad Equation 1$$

The HPLC analytical method was as follows: Chiral HPLC column: CHIRALCEL® OJ-3R (150×4.6 millimeters (mm) inside diameter, 3.0 microns (pm)); temperature: 30° C.; flow rate: 0.625 mL/minute; isocratic 50:50 0.1% formic acid in acetonitrile-0.1% formic acid in water; UV detector at 220 nanometers (nm). Expected elution time of the (S,S)-ester at 18.0 minutes and of the (R,R)-ester at 18.8 minutes.

### Example 3b

### Isolation of (1R,3R)-3-(3,5-bis(trifluoromethyl)phenyl)-2,2-dichlorocyclopropane-1-carboxylic acid (S3a-1)

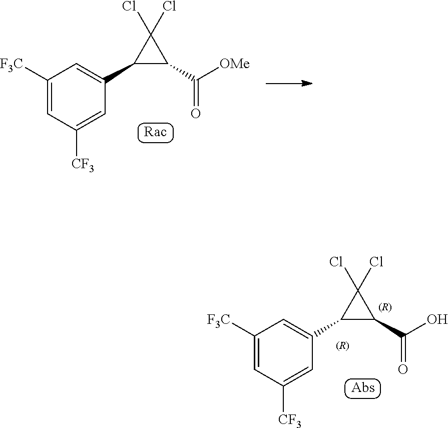

In a 1-L jacketed reactor vessel equipped with a mechanical stirrer with pitched blade type impeller, 0.2 M 2-amino-2-(hydroxymethyl)propane-1,3-diol buffer solution at pH 7 (131.25 mL) and 100 mg/mL lysine solution (70 mL) were added. While mixing at 200 RPM, a solution of trans-rac-methyl 3-(3,5-bis(trifluoromethyl)phenyl)-2,2-dichlorocyclopropane-1-carboxylate (S2a-1; 7 g, 771 mmol) in acetone (79 mL) was added to the reactor. The temperature of the vessel contents was increased to 30° C. and was mixed for 30 minutes. Pseudomonas stutzeri lipase (available from Almac Group product code AH-04; 70 mg) dissolved in deionized water (35 mL) was added to the reactor. The reaction mixture was stirred at 250 RPM and 30° C. for 24 hours.

The reaction solution was harvested into a 1-L centrifuge bottle and centrifuged at 4402 Relative Centrifugal Force (“RCF”) for 5 minutes in a Beckman Avanti J-26 floor centrifuge. The supernatant containing the vast majority of the S3a-1 product was decanted away from the denser oil phase. The denser oil phase largely comprising of unreacted S2a-1 was transferred to 50 mL polypropylene conical vials and centrifuged at 3100 RCF in a Thermo Scientific Sorvall ST8 for a sufficient duration to effect phase separation in order to recover additional S3a-1. The supernatant was combined with the previous supernatant. A total of 287.55 g of supernatant was recovered from 298.22 g of material harvested from the reactor, with the remainder comprising the S,S-methyl ester rich dense oil phase.

Supernatant (47.75 g) was added to a 250 mL glass beaker. 0.1 normal (N) hydrochloric acid (214.8 g) was added over 1 hour using a Watson Marlow 520SU peristaltic pump at 0.6 RPM. The hydrochloric acid reduced the pH of the solution, thereby reducing the solubility limit of S3a-1 resulting in crystallization. The crystallization slurry was centrifuged in 50 mL conical vials at 3100 RCF in a Thermo Scientific Sorvall ST8 for a sufficient duration to sediment the crystals. The wet crystals were transferred to a glass scintillation vial and dried overnight at 25 mm Hg vacuum pressure and ambient temperature. This yielded 0.20413 g of S3a-1 with 92.0% purity.

### Example 3c

### Isolation of (1R,3R)-3-(3,5-bis(trifluoromethyl)phenyl)-2,2-dichlorocyclopropane-1-carboxylic acid (S3a-1)

After the synthesis of (1R,3R)-3-(3,5-bis(trifluoromethyl)phenyl)-2,2-dichlorocyclopropane-1-carboxylic acid (S3a-1) described in Example 3a, acetone was removed from the reaction mixture by evaporation under reduced pressure. The remaining mixture was made basic to pH 11.5 by addition of aqueous 2 M sodium hydroxide solution. The aqueous mixture was then extracted with methyl tert-butyl ether twice. The remaining aqueous layer was subjected to reactive crystallization, where aqueous 3 M hydrochloric acid solution was added slowly to adjust the pH of the mixture to pH 4.0. The resulting slurry was stirred at room temperature for 5 hours and then filtered through a fritted glass funnel to obtain S3a-1 as off-white solid (42% isolated yield).

### Example 4a

### Screening of Enzymes for Activity and Selectivity for the Hydrolysis of trans-rac-3-(3,5-bis(trifluoromethyl)phenyl)-2,2-dichlorocyclopropane-1-carboxylate (S2a-1) to Synthesize (1R,3R)-3-(3,5-bis(trifluoromethyl)phenyl)-2,2-dichlorocyclopropane-1-carboxylic acid (S3a-1)

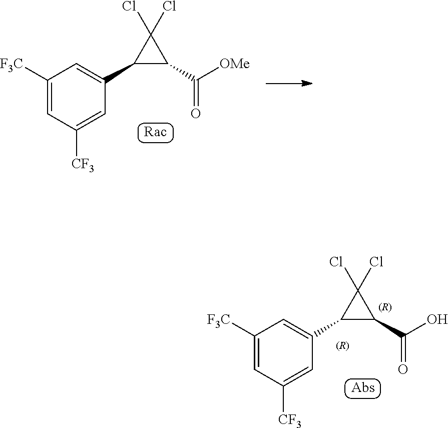

### Reaction:

A solution of trans-rac-methyl 3-(3,5-bis(trifluoromethyl)phenyl)-2,2-dichlorocyclopropane-1-carboxylate (S2a-1) in acetone was prepared to a concentration of 200 milligrams per milliliter (mg/mL; “substrate solution”). A solution of lysine was prepared in water to a concentration of 200 mg/mL (“lysine solution”). A 15-mL conical vial was charged with lysine solution (0.25 mL), substrate solution (1.0 mL), acetone (0.5 mL), and deionized water (3.25 mL). The capped 15-mL conical vial was rotated on a tube inverter for 30 minutes at 40 RPM. Lipases selected from the list shown in Table 4a-2 were weighed into separate 15-mL conical vials (40 mg). Water (1.0 mL) was added to each vial containing lipase. The vials were vortexed for three minutes at 3000 RPM to disperse and partially dissolve the lipase. The contents of the substrate, acetone and lysine solutions were transferred to the vials containing enzyme. The vials were inverted at 40 RPM for 24 hours at 30° C. to allow for the lipase-catalyzed hydrolysis to proceed. After 24 hours two samples were taken for analysis by HPLC to assess approximate conversion of S2a-1 and the enantiomeric excess of the S3a-1 product.

### Analysis of Approximate Conversion by Achiral HPLC:

Well mixed reaction solution (333 μL) was weighed into a 25 mL volumetric flask. An internal standard solution (200 μL) was added to the flask. The internal standard solution consisted of 4,4′-dihydroxydiphenylmethane (8.33 mg) dissolved into acetonitrile (0.5 mL) and water (0.5 mL). Additional 1:1 volume acetonitrile/volume water was added to bring total volume to 25 mL. The solution was vortexed for 30 seconds. An aliquot was transferred into an HPLC vial and analyzed by HPLC using the following method: HPLC column: Agilent ZORBAX SB-Phenyl (150 mm×4.6 mm internal diameter with 3.5 μm particle size, Agilent Part Number 863953-912); temperature: ambient (approximately 20° C.); flow rate: 1.0 mL/minute; injection volume: 5 μm; UV detection at 210 nm; solvent gradient according to table below:

TABLE 4a-1

| Time | Water + 0.03% phosphoric | Acetonitrile |
|---|---|---|
| (min) | acid (volume %) | (volume %) |
| 0.0 | 60 | 40 |
| 8.0 | 5 | 95 |

The internal standard is expected to elute at 3.9 minutes, S3a-1 at 6.2 minutes and S2a-1 at 7.3 minutes. The approximate conversion was calculated from the achiral HPLC results using quantification based on the internal standard and the equation:

$$Approximate Conversion ( \% ) = 100 * \frac{Weight Percent of Acid in Solution}{\begin{matrix}Weight Percent of Acid in Solution + \\ Weight Percent of Ester in Solution\end{matrix}}$$

### Analysis of Enantiomeric Excess by Chiral HPLC:

A 50-μL sample was taken from the conical reaction vial and placed into a filtering HPLC vial to which acetonitrile (400 μL) was added. The filter plunger was inserted and the filtered solution was analyzed by HPLC using the following method: Chiral HPLC column: DAICEL CHIRALCEL® OJ-3R (150 mm×4.6 mm internal diameter, 3 μm silica gel); temperature: 30° C.; flow rate: 0.625 mL/minute; isocratic 50%:50% 0.1% formic acid in water-0.1% formic acid in acetonitrile; UV detection at 220 nm; injection volume of 5.0 μL. Expected elution time of the (S,S)-acid at 7.2 minutes, the (R,R)-acid at 7.7 minutes, the (S,S)-ester at 18.0 minutes and the (R,R)-ester at 18.8 minutes. Enantiomeric excess was calculated from chiral HPLC results using the following equation.

$$Enantiomeric Excess ( ee ) = 100 * [ \frac{( R , R ) Acid Peak Area - ( S , S ) Acid Peak Area}{( R , R ) Acid Peak Area + ( S , S ) Acid Peak Area} ]$$

TABLE 4a-2
Summary of hydrolase activity data

| | Almac | Approximate | Enantiomeric |
|---|---|---|---|
| Carboxylic ester | Group | Conversion | Excess (ee) of |
| hydrolase | Product | (% of total | S3a-1 Product |
| Common Name | Number | S2a-1) | (%) |
| Lipase C from | AH-03 | 36.87% | 97.9% |
| Alcaligenes sp. | | | |
| Lipase from | AH-04 | 48.99% | 98.5% |
| Pseudomonas stutzeri | | | |
| Lipase from | AH-05 | 23.77% | 97.1% |
| Pseudomonas cepacia | | | |
| Lipase E from | AH-08 | 30.04% | 97.6% |
| Alcaligenes sp. | | | |
| Lipase A from | AH-36 | 24.84% | 97.2% |
| Burkholderia cepacia | | | |
| Lipase B from | AH-37 | 22.85% | 96.5% |
| Burkholderia cepacia | | | |
| Lipase B from | AH-42 | 1.29% | 25.3% |
| Candida antarctica | | | |
| Lipase A from | AH-43 | 3.14% | 11.3% |
| Candida antarctica | | | |
| Lipase B from Candida | AH-24 | 0.97% | 6.1% |
| rugosa (Diutina rugosa) | | | |
| Lipase from | AH-27 | 0.77% | 7.3% |
| Aspergillus niger | | | |
| Lipase from | AH-28 | 1.61% | 1.7% |
| Penicillium roquefort | | | |
| Lipase from | AH-32 | 1.27% | 0.6% |
| Penicillium camembertii | | | |

### Example 4b

### Assessing Activity and Selectivity of Pseudomonas Stutzeri Lipase for the Hydrolysis of trans-rac-methyl 2,2-dichloro-3-(3,5-dichlorophenyl)cyclopropane-1-carboxylate (S2a-4) to Synthesize (1R,3R)-2,2-dichloro-3-(3,5-dichlorophenyl)cyclopropane-1-carboxylic acid (S3a-4)

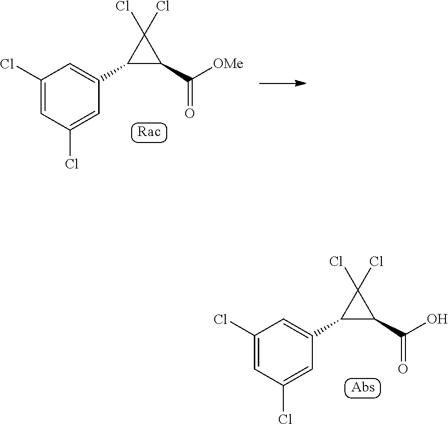 [SMILES: COC(=O)[C@H]1[C@H](c2cc(Cl)cc(Cl)c2)C1(Cl)Cl ; O=C(O)[C@H]1[C@H](c2cc(Cl)cc(Cl)c2)C1(Cl)Cl]

### Reaction:

A solution of lysine was prepared in water to a concentration of 80 mg/mL (“lysine solution”). Four 15-mL conical vials were charged with 200 mg of trans-rac-methyl 2,2-dichloro-3-(3,5-dichlorophenyl)cyclopropane-1-carboxylate (S2a-4), lysine solution (0.5 mL), and one of the following solvents: acetone (2.4 mL), dimethyl sulfoxide (0.45 mL), acetonitrile (0.6 mL) or methyl tert-butyl ether (3 mL). The vial fill volumes were brought to a total of 5 mL with deionized water. The capped 15-mL conical vials were rotated on a tube inverter for 30 minutes at 40 RPM. A lipase solution was prepared by dissolving Pseudomonas stutzeri lipase (Almac Group, AH-04) in deionized water to a concentration of 2 mg/mL (“lipase solution”). Lipase solution (1.0 mL) was added to each 15-mL conical vial. The 15-mL conical vials were inverted at 40 RPM for 24 hours at 30° C. to allow for the lipase catalyzed hydrolysis to proceed.

After 24 hours, 1 N hydrochloric acid (0.25 mL) was added to each of the 15-mL conical vials. The mixture was extracted twice with dichloromethane by adding 3 mL of dichloromethane, vortexing, settling, and removing the dichloromethane by pipetting. The process was repeated for the second extraction. The dichloromethane extracts were combined, dried over sodium sulfate, filtered and concentrated by rotary evaporation. The resulting oil was dissolved in acetonitrile and analyzed by HPLC to assess Approximate Conversion of S2a-4 and by chiral HPLC to access the enantiomeric excess of the S3a-4 product. The analytical results are shown in Table 4b-1.

TABLE 4b-1
Results of enzymatic hydrolysis of S2a-4
to S3a-4 using various co-solvents

| | 40% | 7.5% | 10% | 50% |
|---|---|---|---|---|
| Co-solvent | acetone | DMSO | acetonitrile | MTBE |
| Approximate Conversion | 29.5% | 12.5% | 5.9% | 5.6% |
| Enantiomeric Excess (% ee) | 95.8% | 82% | 67.4% | 84.6% |

### Example 4c

### Assessing Activity and Selectivity of Pseudomonas Stutzeri Lipase for the Hydrolysis of trans-rac-methyl 2,2-dichloro-3-(3-chloro-4-fluorophenyl)cyclopropane-1-carboxylate (S2a-5) to Synthesize (1R,3R)-2,2-dichloro-3-(3-chloro-4-fluorophenyl)cyclopropane-1-carboxylic acid (S3a-5)

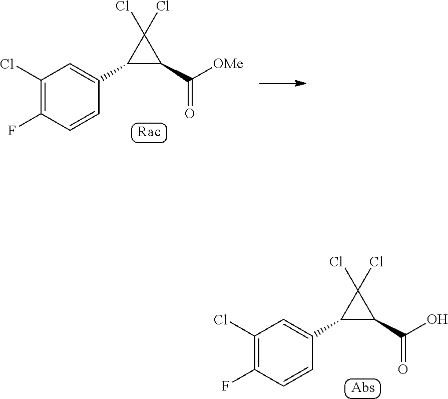 [SMILES: COC(=O)[C@H]1[C@H](c2ccc(F)c(Cl)c2)C1(Cl)Cl ; O=C(O)[C@H]1[C@H](c2ccc(F)c(Cl)c2)C1(Cl)Cl]

A solution of trans-rac-methyl 2,2-dichloro-3-(3-chloro-4-fluorophenyl)cyclopropane-1-carboxylate (S2a-5) in acetone was prepared to a concentration of 200 mg/mL (“substrate solution”). A solution of lysine was prepared in water to a concentration of 80 mg/mL (“lysine solution”). A 15-mL conical vial was charged with lysine solution (0.5 mL), substrate solution (1.0 mL), acetone (1.4 mL), and deionized water (2.1 mL). The capped 15-mL conical vial was rotated on a tube inverter for 30 minutes at 40 RPM. A lipase solution was prepared by dissolving Pseudomonas stutzeri lipase (Almac Group, AH-04) in deionized water to a concentration of 2 mg/mL (“lipase solution”). Lipase solution (1.0 mL) was added to each 15-mL conical vial. The vial was inverted at 40 RPM for 24 hours at 30° C. to allow for the lipase catalyzed hydrolysis to proceed.

To the reaction solution, 1 N hydrochloric acid (0.25 mL) was added. The mixture was extracted twice with dichloromethane by adding 3 mL of dichloromethane, vortexing, settling, and removing the dichloromethane by pipetting. The process was repeated for the second extraction. The dichloromethane extracts were combined, dried over sodium sulfate, filtered and concentrated by rotary evaporation. The resulting oil was dissolved in acetonitrile and analyzed by HPLC to assess Approximate Conversion of S2a-5 and by chiral HPLC to assess the enantiomeric excess of the S3a-5 product. The HPLC analysis showed 26% conversion and the chiral HPLC results showed 94.4% enantiomeric excess of the reaction product.

### Example 5

### Synthesis of (1R,3R)-2,2-dichloro-3-(3-trifluoromethyl-4-fluorophenyl)-2,2-dichlorocyclopropane-1-carboxylic acid (S3a-2)

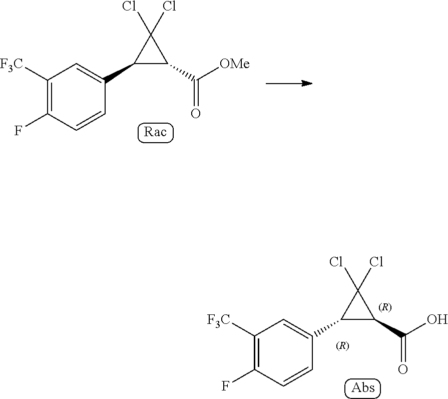

In 100 mL flask, trans-rac-methyl 2,2-dichloro-3-(4-fluoro-3-(trifluoromethyl)phenyl)cyclopropane-1-carboxylate (S2a-2; 500 mg, 1.510 mmol) was dissolved in DMSO (10 mL) and buffer solution (0.1 M potassium phosphate dibasic and monobasic, pH 7.0; 100 mL) at room temperature. The enzyme (Pseudomonas stutzeri lipase, available from Almac Group product code AH-04; 250 mg) was added, and the suspension was stirred at 30° C. for 4 days. The reaction mixture was diluted with ethyl acetate and 6 N hydrochloric acid. The organic layer mostly containing ethyl acetate was separated and was washed with additional water. The organic layer was concentrated by evaporation under vacuum to afford (1R,3R)-2,2-dichloro-3-(4-fluoro-3-(trifluoromethyl)phenyl)cyclopropane-1-carboxylic acid (150 mg, 31.3% isolated molar yield, 96% enantiomeric excess): ¹H NMR (400 MHz, Chloroform-d) δ 7.60-7.41 (m, 2H), 7.30-7.17 (m, 1H), 3.50 (d, J=8.2 Hz, 1H), 2.89 (d, J=8.3 Hz, 1H); ¹⁹F NMR (376 MHz, DMSO) δ −61.48, −114.25; LCMS m/z=316 ([M−H]⁻). Chiral HPLC method: Column: CHIRALPAK@ ZWIX(+), particle size 3 μm, dimension 3 mm×150 mm, DAIC 511584; Mobile phase: 49% acetonitrile-49% methanol-water with 50 millimolar (mM) formic acid and diethylamine; Flow rate: 0.5 mL/minute; Elution time: 9 minutes; Temperature: 25° C.

### Example 6

### Synthesis of (1R,3R)-2,2-dichloro-3-(3,4-dichlorophenyl)cyclopropane-1-carboxylic acid (S3a-3)

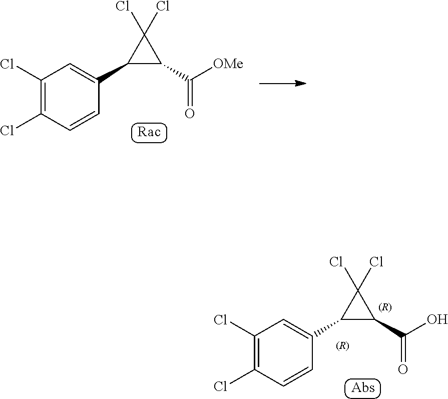

trans-rac-Methyl 2,2-dichloro-3-(3,4-dichlorophenyl)cyclopropane-1-carboxylate (S2a-3; ˜5 mg) in DMSO (50 μL) was added to each of four 2 mL Eppendorf tubes. Solutions of Pseudomonas stutzeri lipase (available from Almac Group product code AH-04;) were prepared in 0.1 M potassium phosphate buffer with pH of 7.0. A sufficient volume of lipase solution and potassium phosphate buffer solution was prepared to add between 0.05 mg and 15 mg of lipase in buffer (950 μL) to each 2 mL Eppendorf tube bringing the total volume in each tube to 1 mL. The reaction mixtures were allowed to proceed using a tube inverter to rotate the vials at 40 RPM at 30° C. for between 40 and 80 hours, according to Table 6-1. The mixture was diluted with ethyl acetate and 6 N hydrochloric acid. The organic layer containing mostly ethyl acetate was collected and analyzed by HPLC. The resulting conversion and enantiomeric excess of each reaction is shown in Table 6-1.

Chiral HPLC method: Column: CHIRALPAK@ ZWIX(+), particle size 3 μm, dimension 3 mm×150 mm, DAIC 511584; Mobile phase: 49% acetonitrile-49% methanol-water with 50 mM formic acid and diethylamine; Flow rate: 0.5 mL/minute; Elution time: 9 minutes; Temperature: 25° C.

TABLE 6-1
Summary of Pseudomonas stutzeri lipase (available from Almac Group
product code AH-04) resolution of S2a-3 to S3a-3

| | | | Loading | | | | Enantio- |
|---|---|---|---|---|---|---|---|
| | | | of | Total | | | meric |
| | Mass | Mass | Lipase* | Re- | Re- | Pro- | Excess (ee) |
| | of | of | (wt % | action | action | duction | of S3a-1 |
| Re- | S2a-3 | Lipase* | ratio to | Volume | Time | Value | Product |
| action | (mg) | (mg) | S2a-3) | (mL) | (hours) | (%)** | (%) |
| 1 | 5 | 15 | 300 | 1 | 40 | 50 | 97 |
| 2 | 5 | 5 | 100 | 1 | 40 | 34 | 97 |
| 3 | 5 | 0.5 | 10 | 1 | 80 | 18 | 97 |
| 4 | 5 | 0.05 | 1 | 1 | 60 | 1 | Not |
| | | | | | | | Determined |

Lipase* = Pseudomonas stutzeri lipase (Almac AH-04)
**Production value was calculated as follows:

$$production value ( \% ) = 100 * 0.5 * ( 1 - \frac{Area units of ( R , R ) ester}{Area units of ( S , S ) ester} )$$

### Example 7

### Synthesis of (1R,3R)-3-(3,5-bis(trifluoromethyl)phenyl)-2,2-dichlorocyclopropane-1-carboxylic acid (S3a-1)

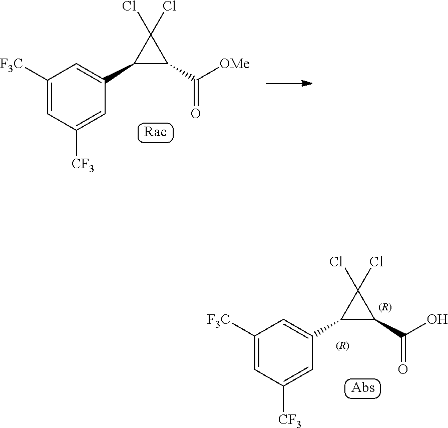

A solution of trans-rac-methyl 3-(3,5-bis(trifluoromethyl)phenyl)-2,2-dichlorocyclopropane-1-carboxylate (S2a-1) in acetone was prepared to a concentration of 200 mg/mL (“substrate solution”). A solution of lysine was prepared in water to a concentration of 100 mg/mL (“lysine solution”). A solution of Pseudomonas stutzeri lipase (available from Almac Group product code AH-04) in water was prepared to a concentration of 2 mg/mL (“enzyme solution”). A 15-mL conical vial was charged with lysine solution (0.25 mL), ester solution (0.5 mL), acetone (1 mL), and water (4.125 mL). The capped 15-mL conical vial was rotated on a tube inverter for 30 minutes at 40 RPM. Enzyme solution (0.125 mL) was added to the 15-mL conical vial, making the mass of Pseudomonas stutzeri lipase equal to 0.25% of the mass of the S2a-1. The tube was inverted at 40 RPM for 48 hours at 30° C. to allow for the lipase resolution to proceed. A 50-μL sample was taken from the conical vial and placed into a filtering HPLC vial to which acetonitrile (400 μL) was added. The filter plunger was inserted, and the filtered solution was analyzed by HPLC.

The HPLC analytical method was as follows: Chiral HPLC column: DAICEL CHIRALCEL® OJ-3R (150 mm×4.6 mm internal diameter, 3 μm silica gel); temperature: 30° C.; flow rate: 0.625 mL/minute; isocratic 50:50 0.1% formic acid in water-0.1% formic acid in acetonitrile; UV detection at 220 nm; injection volume of 5.0 μL. Expected elution time of the (S,S)-acid at 7.2 minutes, the (R,R)-acid at 7.7 minutes, the (S,S)-ester at 18.0 minutes and the (R,R)-ester at 18.8 minutes.

HPLC analysis showed a production value of 47.7% with an enantiomeric excess of 98.8% Production value was calculated as follows.

$$production value ( \% ) = 100 * 0.5 * ( 1 - \frac{Area units of ( R , R ) ester}{Area units of ( S , S ) ester} )$$

### Example 8

### Synthesis of (1R,3R)-3-(3,5-bis(trifluoromethyl)phenyl)-2,2-dichlorocyclopropane-1-carboxylic acid (S3a-1) from trans-rac-ethyl-3-(3,5-bis(trifluoromethyl)phenyl)-2,2-dichlorocyclopropane-1-carboxylate (S2a-6)

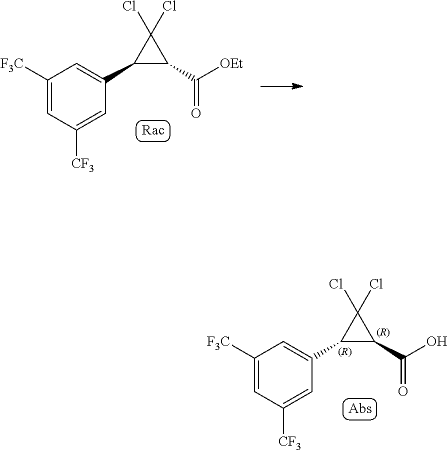

A solution of trans-rac-ethyl-3-(3,5-bis(trifluoromethyl)phenyl)-2,2-dichlorocyclopropane-1-carboxylate (S2a-6) in acetone was prepared to a concentration of 200 mg/mL (“substrate solution”). A solution of anhydrous lysine was prepared in water to a concentration of 80 mg/mL (“lysine solution”). A solution of Pseudomonas stutzeri lipase (available from Meito Sangyo Co., Ltd., product code Lipase TL) in water was prepared to a concentration of 2 mg/mL (“lipase solution”). Two 15-mL conical vials were each charged with lysine solution (0.5 mL), substrate solution (1.0 mL), acetone (0.5 mL), and water (3.0 mL). The capped 15-mL conical vials were rotated on a tube inverter for 30 minutes at 40 RPM. Lipase solution (1.0 mL) was added to one 15-mL conical vial, making the mass of Pseudomonas stutzeri lipase equal to 1.0% of the mass of the S2a-6. The tubes were inverted at 40 RPM for 24 hours at 30° C. A 50-μL sample was taken from each the conical vial and placed into a filtering HPLC vial to which acetonitrile (400 μL) was added. The filter plunger was inserted, and the filtered solution was analyzed by HPLC.

The HPLC analytical method was as follows: Chiral HPLC column: DAICEL CHIRALCEL® OJ-3R (150 mm×4.6 mm internal diameter, 3 μm silica gel); temperature: 30° C.; flow rate: 0.625 mL/minute; isocratic 50:50 0.1% trifluoroacetic acid in water-0.1% trifluoroacetic acid in acetonitrile; UV detection at 220 nm; injection volume of 5.0 μL. Expected elution time of the (S,S)-acid at 7.2 minutes, the (R,R)-acid at 7.7 minutes, the (S,S)-ester at 18.0 minutes and the (R,R)-ester at 18.8 minutes.

Enantiomeric excess was calculated from chiral HPLC results using the equation.

$$Enantiomeric Excess ( ee ) = 100 * [ \frac{( R , R ) Acid Peak Area - ( S , S ) Acid Peak Area}{( R , R ) Acid Peak Area + ( S , S ) Acid Peak Area} ]$$

Production value was calculated from chiral HPLC results using the equation:

$$Production Value ( \% ) = 100 * 0.5 * ( 1 - \frac{Area units of ( R , R ) ester}{Area units of ( S , S ) ester} )$$

The results are presented in Table 8-1.

TABLE 8-1
Production value and enantiomeric excess using trans-
rac-ethyl-3-(3,5-bis(trifluoromethyl)phenyl)-2,2-dichlorocyclopropane-
1-carboxylate (S2a-6) substrate

| | Production | Enantiomeric Excess |
|---|---|---|
| | Value | (ee) of S3a-1 Product |
| Enzyme | (%) | (%) |
| Lipase TL | 47.8% | >99% |

### Example 9

### Use of Pseudomonas Stutzeri Lipase for the Stereoselective Hydrolysis of trans-rac-3-(3,5-bis(trifluoromethyl)phenyl)-2,2-dichlorocyclopropane-1-carboxylate (S2a-1) to Synthesize (1R,3R)-3-(3,5-bis(trifluoromethyl)phenyl)-2,2-dichlorocyclopropane-1-carboxylic acid (S3a-1)

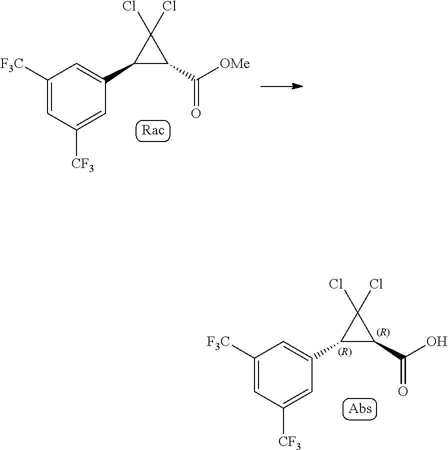

A solution of trans-rac-methyl-3-(3,5-bis(trifluoromethyl)phenyl)-2,2-dichlorocyclopropane-1-carboxylate (S2a-1) in acetone was prepared to a concentration of 200 mg/mL (“substrate solution”). A solution of anhydrous lysine was prepared in water to a concentration of 80 mg/mL (“lysine solution”). A solution of Pseudomonas stutzeri lipase (available from Meito Sangyo Co., Ltd., product code Lipase TL) in water was prepared to a concentration of 2 mg/mL (“Lipase TL solution”). A solution of Pseudomonas stutzeri lipase (available from Almac Group, product code AH-04) in water was prepared to a concentration of 2 mg/mL (“AH-04 solution”). Two 15-mL conical vials were each charged with lysine solution (1.0 mL), substrate solution (1.0 mL), acetone (0.5 mL), and water (2.5 mL). The capped 15-mL conical vials were rotated on a tube inverter for 30 minutes at 40 RPM. Lipase solution (1.0 mL) was added to one 15-mL conical vial, making the mass of Pseudomonas stutzeri lipase equal to 1.0% of the mass of the S2a-1. Esterase solution (1.0 mL) was added to the other 15-mL conical vial, making the mass of esterase equal to 1.0% of the mass of the S2a-1. The tubes were inverted at 40 RPM for 24 hours at 30° C. A 50-μL sample was taken from each of the conical vials and placed into a filtering HPLC vial to which acetonitrile (400 μL) was added. The filter plunger was inserted, and the filtered solution was analyzed by HPLC.

The HPLC analytical method was as follows: Chiral HPLC column: DAICEL CHIRALCEL® OJ-3R (150 mm×4.6 mm internal diameter, 3 μm silica gel); temperature: 30° C.; flow rate: 0.625 mL/minute; isocratic 50:50 0.1% trifluoroacetic acid in water-0.1% trifluoroacetic acid in acetonitrile; UV detection at 220 nm; injection volume of 5.0 μL. Expected elution time of the (S,S)-acid at 7.2 minutes, the (R,R)-acid at 7.7 minutes, the (S,S)-ester at 18.0 minutes and the (R,R)-ester at 18.8 minutes.

Enantiomeric excess was calculated from chiral HPLC results using the equation.

$$Enantiomeric Excess ( ee ) = 100 * [ \frac{( R , R ) Acid Peak Area - ( S , S ) Acid Peak Area}{( R , R ) Acid Peak Area + ( S , S ) Acid Peak Area} ]$$

Production value was calculated from chiral HPLC results using the equation:

$$Production Value ( \% ) = 100 * 0.5 * ( 1 - \frac{Area units of ( R , R ) ester}{Area units of ( S , S ) ester} )$$

The results are presented in Table 9-1.

TABLE 9-1
Production value and enantiomeric excess using trans-
rac-methyl-3-(3,5-bis(trifluoromethyl)phenyl)-2,2-dichlorocyclopropane-
1-carboxylate (S2a-1) substrate

| | Production | Enantiomeric Excess |
|---|---|---|
| | Value | (ee) of S3a-1 Product |
| Enzyme | (%) | (%) |
| Lipase TL | 48.1 | 98.7% |
| AH-04 | 48.9 | 98.6% |

### Example 10

### Use of Pseudomonas Fluorescens Lipase for the Stereoselective Hydrolysis of trans-rac-3-(3,5-bis(trifluoromethyl)phenyl)-2,2-dichlorocyclopropane-1-carboxylate (S2a-1) to Synthesize (1R,3R)-3-(3,5-bis(trifluoromethyl)phenyl)-2,2-dichlorocyclopropane-1-carboxylic acid (S3a-1)

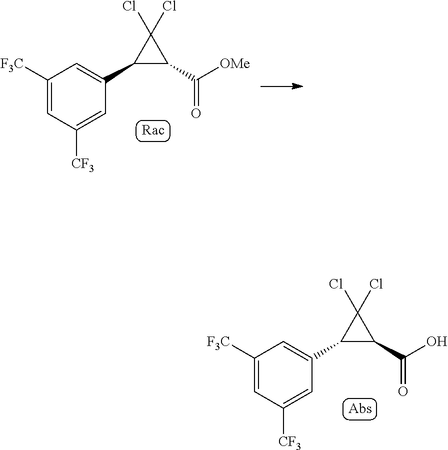 [SMILES: COC(=O)[C@@H]1[C@@H](c2cc(C(F)(F)F)cc(C(F)(F)F)c2)C1(Cl)Cl ; O=C(O)[C@H]1[C@H](c2cc(C(F)(F)F)cc(C(F)(F)F)c2)C1(Cl)Cl]

### Reaction:

A solution of trans-rac-methyl-3-(3,5-bis(trifluoromethyl)phenyl)-2,2-dichlorocyclopropane-1-carboxylate (S2a-1) in acetone was prepared to a concentration of 100 mg/mL (“substrate solution”). A solution of lysine was prepared in water to a concentration of 100 mg/mL (“lysine solution”) using anhydrous lysine as the starting material. A solution of monobasic potassium phosphate and dibasic potassium phosphate was prepared in water to a concentration of 0.1 M (“buffer solution”) in a ratio to result in a pH of 7.0. A 15 mL conical vial was charged with lysine solution (1.0 mL), substrate solution (1.0 mL), acetone (0.5 mL), and buffer solution (2.5 mL). The capped 15 mL conical vial was rotated on a tube inverter for 30 minutes at 40 RPM. A solution of Pseudomonas fluorescens lipase (AH-35, Almac Group) was prepared by dissolving to a concentration of 20 mg/mL (“enzyme solution”) in buffer solution. To the 15 mL conical vial was added 1 mL of the enzyme solution. The vial was inverted at 40 RPM for 24 hours at 30° C. to allow for the enzyme-catalyzed hydrolysis to proceed. After 24 hours, a sample was taken for analysis by HPLC to assess the production value and the enantiomeric excess of the S3a-1 product.

### Analysis by Chiral HPLC:

A 50-μL sample was taken from the conical reaction vial and placed into a filtering HPLC vial to which acetonitrile (400 μL) was added. The filter plunger was inserted and the filtered solution was analyzed by HPLC using the following method: Chiral HPLC column: DAICEL CHIRALCEL® OJ-3R (150 mm×4.6 mm internal diameter, 3 μm silica gel); temperature: 30° C.; flow rate: 0.625 mL/minute; isocratic 50%:50% 0.1% trifluoroacetic acid in water-0.1% trifluoroacetic acid in acetonitrile; UV detection at 220 nm; injection volume of 5.0 μL. Expected elution time of the (S,S)-acid at 7.2 minutes, the (R,R)-acid at 7.7 minutes, the (S,S)-ester at 18.0 minutes and the (R,R)-ester at 18.8 minutes.

Enantiomeric excess was calculated from chiral HPLC results using the equation.

$$Enantiomeric Excess ( ee ) = 100 * [ \frac{( R , R ) Acid Peak Area - ( S , S ) Acid Peak Area}{( R , R ) Acid Peak Area + ( S , S ) Acid Peak Area} ]$$

Production value was calculated from chiral HPLC results using the equation:

$$Production Value ( \% ) = 100 * 0.5 * ( 1 - \frac{Area units of ( R , R ) ester}{Area units of ( S , S ) ester} )$$

Analysis of the reaction vial resulted in a production value of 7.9% and an enantiomeric excess (ee) of 95%.

Consequently, in light of the above the following additional, non-exhaustive, disclosure details (d) are provided.
1d. A process comprising oxidizing S1a to S1b with an oxidizing agent in the presence of a polar solvent

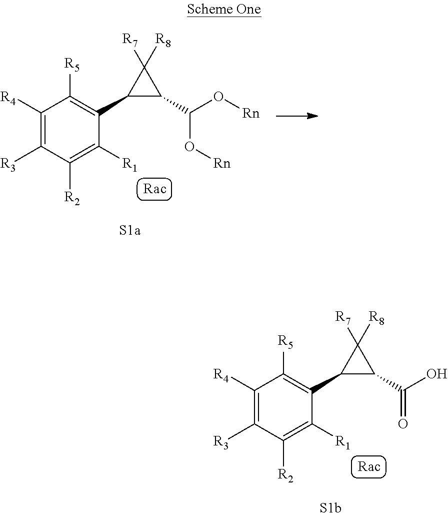

wherein

(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H;

(b) R₇ and R₈ are each independently F, Cl, Br, or I; and

(c) (1) each Rₙ is independently a (C₁-C₆)alkyl, or
(2) both Rₙ form a (C₂-C₆)alkyl link between the two oxygen atoms.
2d. A process according to 1d wherein R₂ and R₄ are CF₃; R₁, R₃, and R₅, are H; R₇ and R₈ are Cl; and each Rₙ is C₂H₅

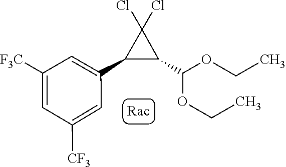 [SMILES: CCOC(OCC)[C@@H]1[C@@H](c2cc(C(F)(F)F)cc(C(F)(F)F)c2)C1(Cl)Cl]

### trans-rac-1-(2,2-dichloro-3-(diethoxymethyl)cyclopropyl)-3,5-bis(trifluoromethyl)benzene

3d. A process according to 1d or 2d wherein said oxidizing agent is oxygen, sodium hypochlorite, ozone, hydrogen peroxide, organic peracids, potassium peroxymonosulfate, potassium persulfate, potassium hydrogen peroxymonosulfate sulfate, or mixtures thereof.
4d. A process according to 3d wherein the amount of oxidizing agent used is from about 0.1 moles to about 3 moles of oxidizing agent per mole of S1a.
5d. A process according to 3d wherein the amount of oxidizing agent used is from about 0.5 moles to about 1.5 moles of oxidizing agent per mole of S1a.
6d. A process according to any of the previous details wherein said polar solvent is a polar aprotic solvent.
7d. A process according to any of the previous details wherein said polar solvent is a polar protic solvent.
8d. A process according to any of the previous details wherein said polar solvent is ethyl acetate, tetrahydrofuran, dichloromethane, acetone, acetonitrile, dimethylformamide, dimethyl sulfoxide, or mixtures thereof.
9d. A process according to any of the previous details wherein said polar solvent is acetic acid, n-butanol, isopropanol, n-propanol, ethanol, methanol, formic acid, tert-butyl alcohol, water, or mixtures thereof.
10d. A process according to any of the previous details wherein said solvent is a mixture of two or more polar solvents wherein said polar solvents are ethyl acetate, tetrahydrofuran, dichloromethane, acetone, acetonitrile, dimethylformamide, acetic acid, n-butanol, isopropanol, n-propanol, ethanol, methanol, formic acid, tert-butyl alcohol, water, mixtures thereof, or a mixture selected from Table S1-MR.
11d. A process according to any of the previous details wherein said process is conducted at a temperature from about 0° C. to about 80° C.
12d. A process according to any of the previous details wherein said process is conducted at a temperature from about 20° C. to about 60° C.
13d. A process according to any of the previous details wherein said process is conducted at a pressure from about 10 kPa to about 1000 kPa.
14d. A process according to any of the previous details wherein said process is conducted at a pressure from about 50 kPa to about 150 kPa.
15d. A process according to any of the previous details wherein said process is conducted in the presence of an acid catalyst.
16d. A process comprising esterifying S1b to S2a in the presence of an acid and an (C₁-C₆)alcohol wherein

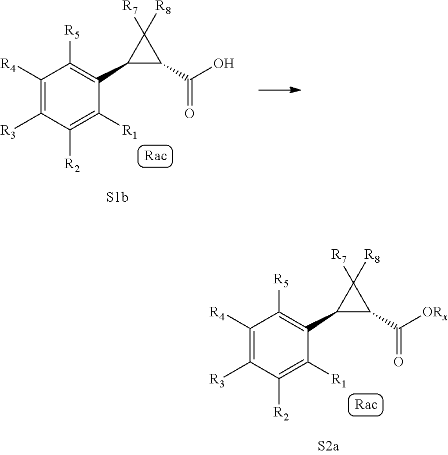

(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H;

(b) R₇ and R₈ are each independently F, Cl, Br, or I;

(c) Rₓ is (C₁-C₆)alkyl or (C₁-C₆)hydroxyalkyl;

optionally wherein said S1b is produced according to 1d through 15d.
17d. A process according to 16d wherein R₂ and R₄ are CF₃; R₁, R₃, and R₅, are H; R₇ and R₈ are Cl; and Rₓ is CH₃

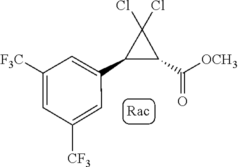 [SMILES: COC(=O)[C@@H]1[C@@H](c2cc(C(F)(F)F)cc(C(F)(F)F)c2)C1(Cl)Cl]

### trans-rac-methyl 3-(3,5-bis(trifluoromethyl)phenyl)-2,2-dichlorocyclopropane-1-carboxylate

17.5d. A process according to 16d wherein R₂ and R₄ are CF₃; R₁, R₃, and R₅, are H; R₇ and R₈ are Cl; and Rₓ is CH₂CH₃

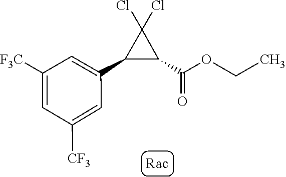 [SMILES: CCOC(=O)[C@@H]1[C@@H](c2cc(C(F)(F)F)cc(C(F)(F)F)c2)C1(Cl)Cl]

### trans-rac-ethyl 3-(3,5-bis(trifluoromethyl)phenyl)-2,2-dichlorocyclopropane-1-carboxylate

18d. A process according to 16d, 17d or 17.5d wherein said acid is sulfuric acid, p-toluenesulfonic acid monohydrate, methanesulfonic acid, scandium(III) triflate, mixtures thereof, or an acid resin.
19d. A process according to 18d wherein about 0.001 moles to about 5 moles of an acid per mole of S1b.
20d. A process according to 18d wherein about 0.05 moles to about 0.5 moles of an acid per mole of S1b.
21d. A process according to any of the previous details 16d through 20d wherein said alcohol is n-butanol, isopropanol, n-propanol, ethanol, methanol, ethylene glycol, tert-butyl alcohol, or mixtures thereof.
22d. A process according to any of the previous details 16d through 21d wherein said alcohol is in a mixture with at least one solvent selected from toluene, carbon tetrachloride, benzene, diethyl ether, hexane, heptane, or dichloromethane.
23d. A process according to any of previous details 16d through 22d wherein the reaction is conducted at temperatures from about 0° C. to about 100° C.
24d. A process according to any of previous details 16d through 23d wherein the reaction is conducted at temperatures about 50° C. to about 70° C.
25d. A process according to any of previous details 16d through 24d wherein the reaction is conducted at pressures from about 10 kPa to about 1000 kPa.
26d. A process according to any of previous details 16d through 25d wherein the reaction is conducted at pressures from about 50 kPa to about 150 kPa.
27d. A process according to any of previous details 16d through 26d wherein a drying agent is used.
28d. A process according to any of previous details 16d through 27d wherein about 0.1 to about 5 moles of drying agents may be used per mole of S1b.
29d. A process according to any of previous details 16d through 27d about 0.2 to about 2 moles of drying agents may be used per mole of S1b.
30d. A process according to any of previous details 16d through 29d wherein said drying agent is an orthoester having the formula R_{y1}C(OR_{y})₃ where R_{y1} is hydrogen or a (C₁-C₆)alkyl, and R_{y2} is (C₁-C₆)alkyl.
31d. A process according to any of previous details 16d through 30d wherein said drying agent is triethyl orthoacetate, CH₃C(OCH₂CH₃)₃; trimethyl orthoacetate, CH₃C(OCH₃)₃; triethyl orthoformate, HC(OCH₂CH₃)₃; trimethyl orthoformate, HC(OCH₃)₃, molecular sieves, magnesium sulfate, calcium chloride, and sodium sulfate or mixtures of drying agents.
32d. A process comprising hydrolyzing S2a to S3a using one or more carboxylic ester hydrolases in the presence of water and optionally an aqueous buffer

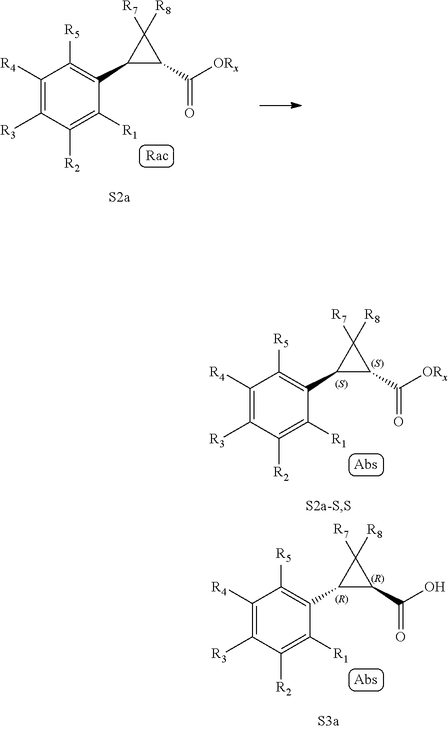

(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H;

(b) R₇ and R₈ are each independently F, Cl, Br, or I;

(c) Rₓ is (C₁-C₆)alkyl or (C₁-C₆)hydroxyalkyl; and

optionally, wherein S2a is produced by 16d through 31d.
33d. A process according to 32d wherein R₂ and R₄ are CF₃; R₁, R₃, and R₅, are H; R₇ and R₈ are Cl; and Rₓ is CH₃

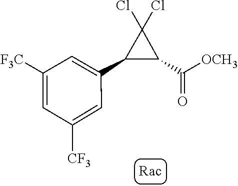 [SMILES: COC(=O)[C@@H]1[C@@H](c2cc(C(F)(F)F)cc(C(F)(F)F)c2)C1(Cl)Cl]

### trans-rac-methyl 3-(3,5-bis(trifluoromethyl)phenyl)-2,2-dichlorocyclopropane-1-carboxylate

34d. A process according to 32d or 33d wherein said one or more carboxylic ester hydrolases is, Pseudomonas stutzeri lipase, Pseudomonas cepacia lipase, Alcaligenes sp. Lipase E, Alcaligenes sp. Lipase C, Pseudomonas fluorencens lipase, Burkholderia cepacia Lipase A, Burkholderia cepacia Lipase B, or a mixture thereof.
35d. A process according to 34d wherein the production value of S2a is greater than 20 percent.
36d. A process according to 34d wherein the production value of S2a is greater than 30 percent.
37d. A process according to 34d wherein the production value of S2a is greater than 40 percent.
38d. A process according to 34d wherein the enantiomeric excess (ee) of S3a, where the (R,R)-enantiomer is greater than 80 percent.
39d. A process according to 34d wherein the enantiomeric excess (ee) of S3a, where the (R,R)-enantiomer is greater than 90 percent.
40d. A process according to 34d wherein the enantiomeric excess (ee) of S3a, where the (R,R)-enantiomer is greater than 95 percent.
41d. A process according to 34d wherein the enantiomeric excess (ee) of S3a, where the (R,R)-enantiomer is greater than 99 percent.
42d. A process according to any of the details 32d through 41d wherein said carboxylic ester hydrolase has a homology of at least 90% compared to one of the following lipases: Pseudomonas stutzeri lipase, Pseudomonas cepacia lipase, Alcaligenes sp. Lipase E, Candida rugosa Lipase B, Pseudomonas fluorencens lipase, Burkholderia cepacia Lipase A, or Burkholderia cepacia Lipase B.
43d. A process according to any of details 32d through 41d wherein said carboxylic ester hydrolase has a homology of at least 95% compared to one of the following carboxylic ester hydrolases: Pseudomonas stutzeri lipase, Pseudomonas cepacia lipase, Alcaligenes sp. Lipase E, Candida rugosa Lipase B, Pseudomonas fluorencens lipase, Burkholderia cepacia Lipase A, or Burkholderia cepacia Lipase B.
44d. A process according to any of 32d through 41d wherein said carboxylic ester hydrolase has a homology of at least 99% compared to one of the following carboxylic ester hydrolases: Pseudomonas stutzeri lipase, Pseudomonas cepacia lipase, Alcaligenes sp. Lipase E, Candida rugosa Lipase B, Pseudomonas fluorencens lipase, Burkholderia cepacia Lipase A, or Burkholderia cepacia Lipase B.
45d. A process according to any of details 32d through 44d wherein the amount of carboxylic ester hydrolase used is from about 0.01% to about 200% by weight with respect to S2a.
46d. A process according to any of details 32d through 44d wherein the amount of carboxylic ester hydrolase used is from about 0.1% to about 5% by weight with respect the S2a.
47d. A process according to any of details 32d through 46d wherein the carboxylic ester hydrolase can be immobilized or supported on a polymer, silica, or other support material.
48d. A process according to any of details 32d through 47d wherein said aqueous buffer is sodium phosphate, 2-[bis(2-hydroxyethyl)amino]-2-(hydroxymethyl)propane-1,3-diol, 2-[4-(2-hydroxyethyl)piperazin-1-yl]ethanesulfonic add, potassium phosphate, 3-morpholinopropane-1-sulfonic acid, piperazine-N,N′-bis(2-ethanesulfonic acid), sodium citrate, 3-{[1,3-dihydroxy-2-(hydroxymethyl)propan-2-yl]amino}propane-1-sulfonic acid, lysine, 2-amino-2-(hydroxymethyl)propane-1,3-diol, or mixtures thereof.
49d. A process according to any of details 32d through 48d wherein the buffer concentration is from about 0.0001 molar to about 0.5 M.
50d. A process according to any of details 32d through 48d wherein the buffer concentration is from 0.05 M to about 0.2 M.
51d. A process according to any of details 32d through 50d wherein the pH of the reaction mixture is about pH 5 to about pH 11.
52d. A process according to any of details 32d through 50d wherein the pH of the reaction mixture is about pH 6 to about pH 10.
53d. A process according to any of details 32d through 52d wherein said process is conducted in the presence of an amine base.
54d. A process according to 53d wherein said amine base is lysine, ethanolamine, glycine, or mixtures thereof.
55d. A process according to 53d or 54d wherein the amount of amine base is from about 0.1 weight percent (wt %) to about 150 weight percent based on the weight of S2a.
56d. A process according to 53d or 54d wherein the amount of amine base is from about 10 weight percent to about 40 weight percent based on the weight of S2a.
57d. A process according to any of details 32d to 56d wherein said reaction is conducted in the presence of a cosolvent wherein said cosolvent is acetone, acetonitrile, methyl tetrahydrofuran, methyl tert-butyl ether, hexane, toluene, methyl ethyl ketone, cyclopentyl methyl ether, dimethyl sulfoxide, dimethoxyethane, or mixtures thereof.
58d. A process according to 57d wherein said reaction is conducted in the presence of a cosolvent and the amount of cosolvent is from about 1% by volume to about 90% of the entire volume based on the volume of the reaction.
59d. A process according to 57d wherein said reaction is conducted in the presence of a cosolvent and the amount of cosolvent is from about 10% by volume to about 40% of the entire volume based on the volume of the reaction.
60d. A process according to any of previous 32d through 59d wherein the reaction is conducted at temperatures from about 0° C. to about 80° C.
61d. A process according to any of previous 32d through 60d wherein the reaction is conducted at temperatures from about 15° C. to about 60° C.
62d. A process according to any of previous 32d through 60d wherein the reaction is conducted at temperatures about 25° C. to about 50° C.
63d. A process according to any of previous 32d through 62d wherein the reaction is conducted at pressures from about 10 kPa to about 1000 kPa.
64d. A process according to any of previous 32d through 62d wherein the reaction is conducted at pressures from about 50 kPa to about 150 kPa.
65d. A molecule useful for the production of arylcyclopropyl carboxylic acids said molecule selected from the group consisting of

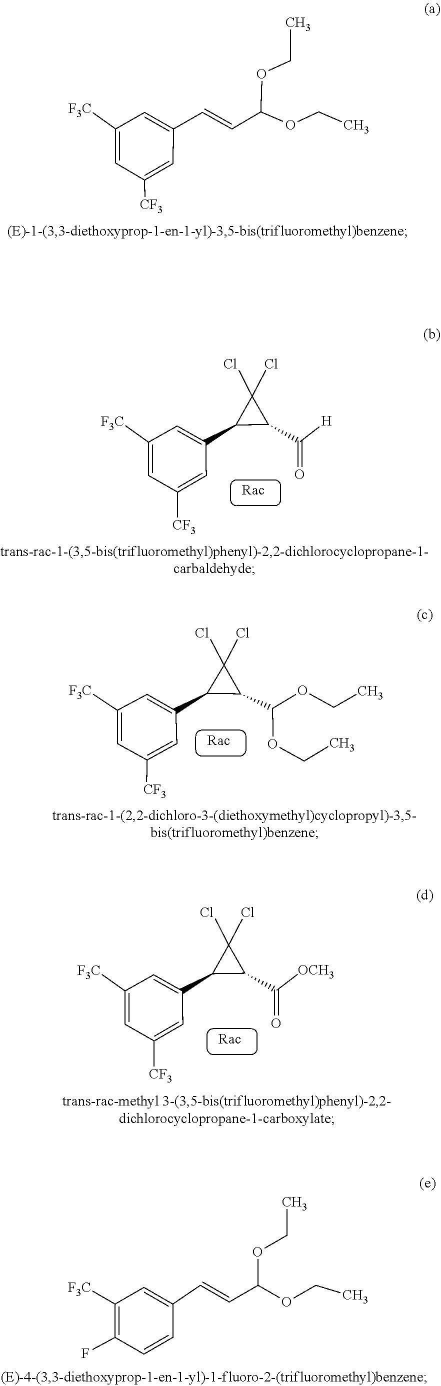 [SMILES: CCOC(/C=C/c1cc(C(F)(F)F)cc(C(F)(F)F)c1)OCC ; O=C[C@@H]1[C@@H](c2cc(C(F)(F)F)cc(C(F)(F)F)c2)C1(Cl)Cl ; CCOC(OCC)[C@@H]1[C@@H](c2cc(CC(F)(F)F)cc(C(F)(F)F)c2)C1(Cl)Cl ; COC(=O)[C@@H]1[C@@H](c2cc(C(F)(F)F)cc(C(F)(F)F)c2)C1(Cl)Cl ; CCOC(/C=C/c1ccc(F)c(C(F)(F)F)c1)OCC]

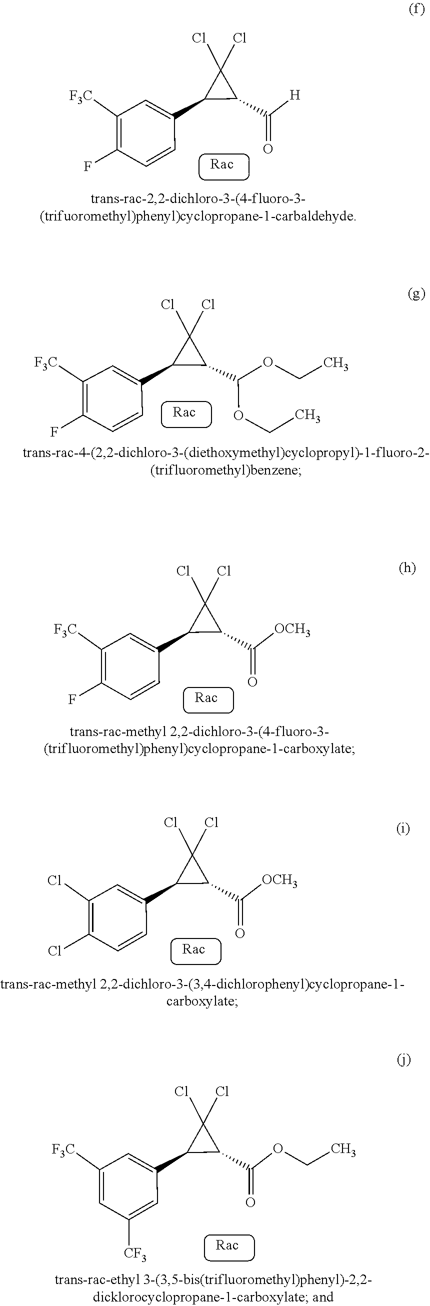 [SMILES: O=C[C@@H]1[C@@H](c2ccc(F)c(C(F)(F)F)c2)C1(Cl)Cl ; CCOC(OCC)[C@@H]1[C@@H](c2ccc(F)c(C(F)(F)F)c2)C1(Cl)Cl ; COC(=O)[C@@H]1[C@@H](c2ccc(F)c(C(F)(F)F)c2)C1(Cl)Cl ; COC(=O)[C@@H]1[C@@H](c2ccc(Cl)c(Cl)c2)C1(Cl)Cl ; CCOC(=O)[C@@H]1[C@@H](c2cc(C(F)(F)F)cc(C(F)(F)F)c2)C1(Cl)Cl]

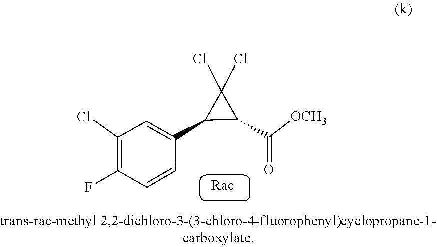 [SMILES: COC(=O)[C@@H]1[C@@H](c2ccc(F)c(Cl)c2)C1(Cl)Cl]

66d. A process comprising the steps of

(A) oxidizing S1a to S1b with an oxidizing agent in the presence of a polar solvent

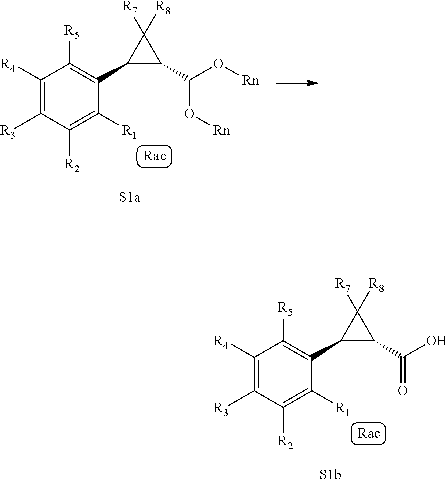

wherein
(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H;
(b) R₇ and R₈ are each independently F, Cl, Br, or I; and
(c) (1) each Rₙ is independently a (C₁-C₆)alkyl, or
   (2) both Rₙ form a (C₂-C₆)alkyl link between the two oxygen atoms,

optionally wherein

any one or more of details 2d, 3d, 4d, 5d, 6d, 7d, 8d, 9d, 10d, 11d, 12d, 13d, 14d, and 15d, is also used in (A);

followed by

(B) esterifying S1b to S2a in the presence of an acid and an (C₁-C₆)alcohol

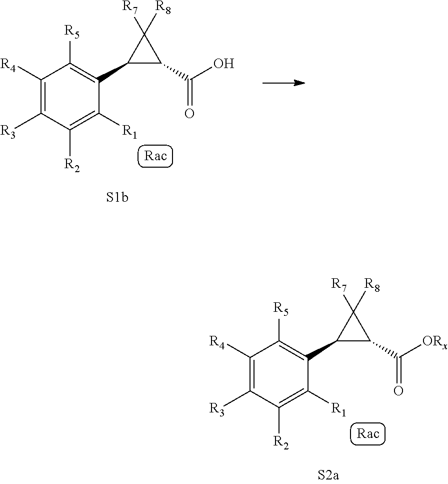

wherein
R₁, R₂, R₃, R₄, R₅, R₇, and R₈ are as above in (A); and
Rₓ is (C₁-C₆)alkyl or (C₁-C₆)hydroxyalkyl

optionally wherein

any one or more of details 17d, 17.5d, 18d, 19d, 20d, 21d, 22d, 23d, 24d, 25d, 26d, 27d, 28d, 29d, 30d, and 31d, is also used in (B);

followed by

(C) hydrolyzing S2a to S3a using one or more carboxylic ester hydrolases in the presence of water and optionally an aqueous buffer

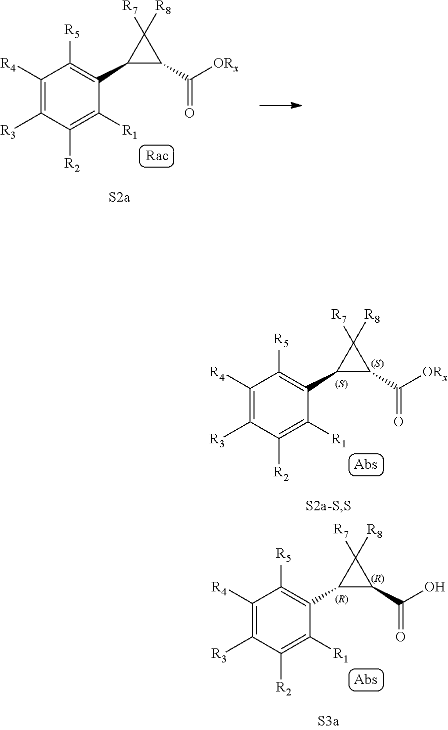

wherein

R₁, R₂, R₃, R₄, R₅, R₇, R₈, and Rₓ are as above in (B)

optionally wherein

any one or more of details 33d, 34d, 35d, 36d, 37d, 38d, 39d, 40d, 41d, 42d, 43d, 44d, 45d, 46d, 47d, 48d, 49d, 50d, 51d, 52d, 53d, 54d, 55d, 56d, 57d, 58d, 59d, 60d, 61d, 62d, 63d, and 64d, is also used in (C).
67d. An enantiomerically enriched preparation of S3a, wherein the enantiomeric excess of the S3a (R,R)-enantiomer is greater than 80%, greater than 90%, or greater than 95%

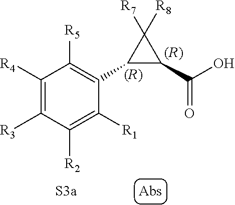

(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H; and

(b) R₇ and R₈ are each independently F, Cl, Br, or I.
68d. The preparation of detail 67d, wherein the enantiomeric excess of the S3a (R,R)-enantiomer is greater than 96%, greater than 97%, or greater than 98%.
69d. A pesticidal formulation comprising the enantiomerically enriched preparation of details 67d or 68d.
70d. A preparation according to details 67d or 68d, wherein R₂ and R₄ are CF₃; R₁, R₃, and R₅ are H; and R₇ and R₈ are Cl

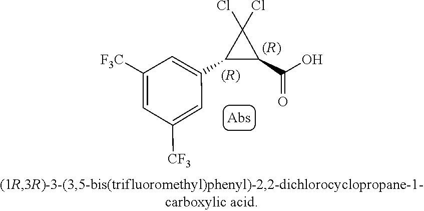

71d. A pesticidal formulation according to detail 69d, wherein R₂ and R₄ are CF₃; R₁, R₃, and R₅ are H; and R₇ and R₈ are Cl

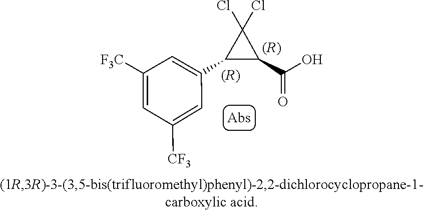

1. A process comprising oxidizing S1a to S1b with an oxidizing agent in the presence of a polar solvent 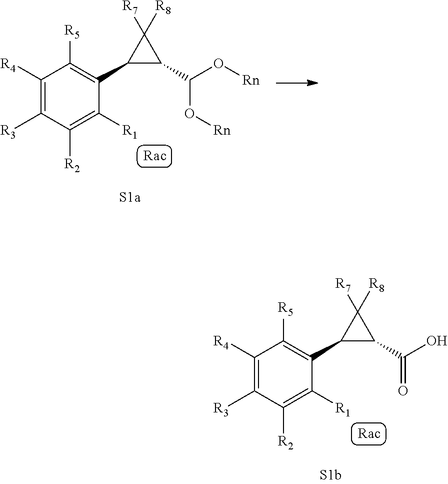
wherein
(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H;
(b) R₇ and R₈ are each independently F, Cl, Br, or I; and
(c) (1) each Rₙ is independently a (C₁-C₆)alkyl, or
(2) both Rₙ form a (C₂-C₆)alkyl link between the two oxygen atoms.

2. A process comprising esterifying S1b to S2a in the presence of an acid and an (C₁-C₆)alcohol 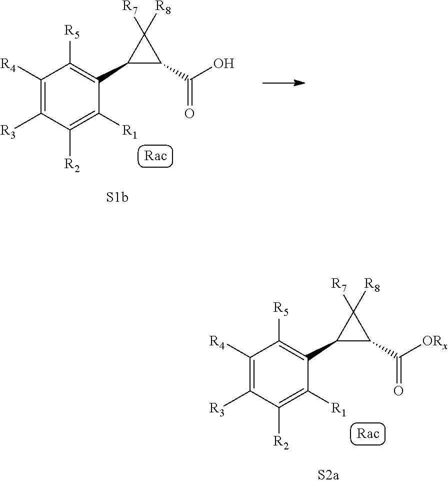
wherein
(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H;
(b) R₇ and R₈ are each independently F, Cl, Br, or I; and
(c) Rₓ is (C₁-C₆)alkyl or (C₁-C₆)hydroxyalkyl.

3. A process comprising hydrolyzing S2a to S3a using one or more carboxylic ester hydrolases in the presence of water and optionally an aqueous buffer 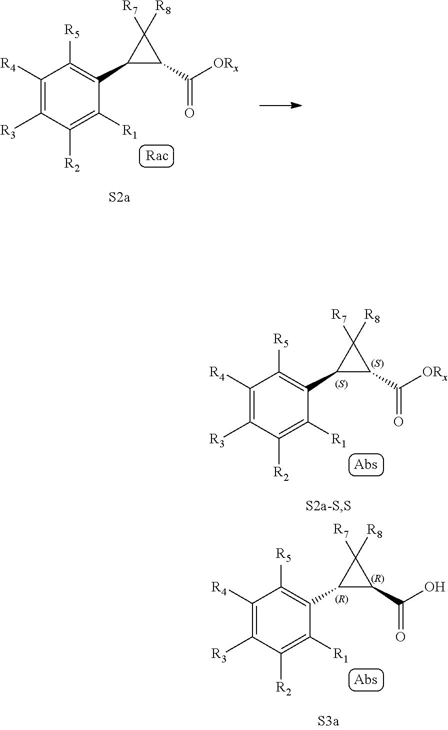
wherein
(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H;
(b) R₇ and R₈ are each independently F, Cl, Br, or I; and
(c) Rₓ is (C₁-C₆)alkyl or (C₁-C₆)hydroxyalkyl.

4. A process comprising the steps of
(A) oxidizing S1a to S1b with an oxidizing agent in the presence of a polar solvent 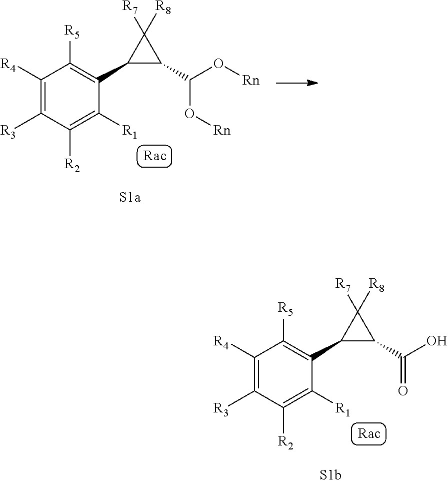
wherein
(a) R₁, R₂, R₃, R₄, and R₅ are each independently H, F, Cl, Br, I, CN, NH₂, NO₂, (C₁-C₆)alkyl, (C₁-C₆)alkoxy, (C₁-C₆)haloalkyl, or (C₁-C₆)haloalkoxy, with the proviso that at least one of R₂, R₃, and R₄ is not H;
(b) R₇ and R₈ are each independently F, Cl, Br, or I; and
(c) (1) each Rₙ is independently a (C₁-C₆)alkyl, or
(2) both Rₙ form a (C₂-C₆)alkyl link between the two oxygen atoms
followed by
(B) esterifying S1b to S2a in the presence of an acid and an (C₁-C₆)alcohol 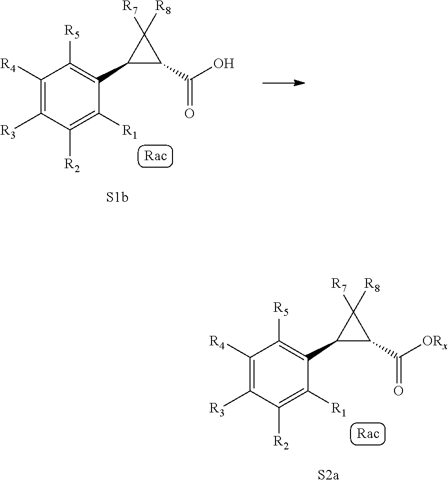
wherein
R₁, R₂, R₃, R₄, R₅, R₇, and R₈ are as above in (A); and
Rₓ is (C₁-C₆)alkyl or (C₁-C₆)hydroxyalkyl;
followed by
(C) hydrolyzing S2a to S3a using one or more carboxylic ester hydrolases in the presence of water and optionally an aqueous buffer 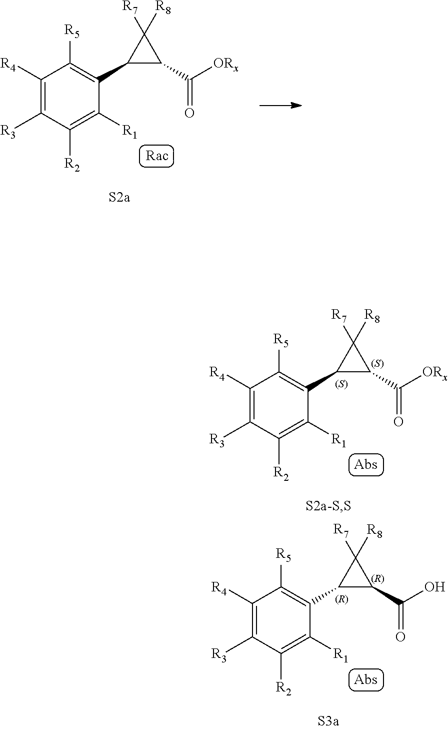
wherein
R₁, R₂, R₃, R₄, R₅, R₇, R₈, and Rₓ are as above in (B).

5. (canceled)

6. (canceled)

7. (canceled)

8. (canceled)

9. (canceled)

10. (canceled)
